(19) United States
(12) Patent Application Publication
(10) Pub. No.: US 2020/0037858 A1
(43) Pub. Date: Feb. 6, 2020

(54) FAIRING FOR FREE SCAR INSTRUMENT AND METHOD

(71) Applicant: Luiz Lanat Pedreira de Cerqueira Filho, Orlando, FL (US)

(72) Inventor: Luiz Lanat Pedreira de Cerqueira Filho, Orlando, FL (US)

(73) Assignee: Luiz Lanat Pedreira de Cerqueira Filho, Salvador (BR)

(21) Appl. No.: 16/510,901

(22) Filed: Jul. 13, 2019

Related U.S. Application Data
(63) Continuation-in-part of application No. 16/458,216, filed on Jul. 1, 2019.
(60) Provisional application No. 62/748,562, filed on Oct. 22, 2018.
(60) Provisional application No. 62/735,169, filed on Sep. 23, 2018.
(60) Provisional application No. 62/698,002, filed on Jul. 13, 2018.
(60) Provisional application No. 62/692,793, filed on Jul. 1, 2018.

(51) Int. Cl. A61B 1/00; A61B 1/04; A61B 17/32; A61B 17/34; A61B 17/115; A61B 18/14; A61B 17/30

(52) U.S. Cl. CPC A61B 1/00131; A61B 1/00154; A61B 1/04; A61B 1/00101; A61B 17/320092; A61B 2018/00601; A61B 17/115; A61B 18/14; A61B 17/30; A61B 17/3478; A61B 2017/320093; A61B 17/3423

(57) ABSTRACT

The inventor provides a surgical device that performs surgery with minimal tissue trauma and is adapted to access the surgical site by a trocar having a plurality of cannulas or by a conventional trocar. The invention including: (a) a plurality of elongated rods, each including a proximal end and a distal end; (b) a holder; (c) a functional element; and (d) a fairing; wherein each said proximal end is connectable to said holder; wherein each said distal end is connectable to said functional element; wherein said fairing removably covers said plurality of elongated rods. Method are provided.

### CROSS-REFERENCE TO RELATED APPLICATIONS

This application claims an invention which was disclosed in a provisional application No. U.S. 62/698,002, filed 2018 Jul. 13, entitled “Fairing for surgical instrument of body divided into stems and method”, and in part of application U.S. Ser. No. 16/458,216, filed 2019-07-01, entitled “Free scar instrument and method”. The benefit under 35 USC § 119(e) of the United States provisional application is hereby claimed, and the afore mentioned application is hereby incorporated herein by reference.

### DESCRIPTION

### Field of Invention

The invention is in the field of medical devices and pertains, particularly, to a minimally invasive surgical device including a firing to perform surgery without scarring and access the surgical site through a conventional trocar.

### Background

In the art of medical devices for performing surgery, many different types of minimally invasive surgical devices have been developed to perform surgery.

One problem with the traditional minimally invasive surgical devices is to have a single body with a thick outer diameter that damages the tissue. Another problem with the traditional minimally invasive devices is to require a thickened outer diameter trocar to provide an access passageway to the surgical site, the large diameter trocar punctures of the prior art cause scar and keloids.

Another limitation of the current minimally invasive device is the need for a tissue incision to access the surgical site, generally, with the use of a scalpel blade that damages the tissue. Another limitation of the current minimally invasive devices is that the tissue incisions required for the passage of the minimally invasive devices usually result in scars.

Minimally invasive surgical devices for performing surgery, such as: robotic surgery, laparoscopic video surgery, thoracoscopic video surgery, video cardiac surgery, video arthroscopic surgery, video urologic surgery, video neurological surgery, video ophthalmologic surgery, orthopedic video surgery, and others, damages the tissue and causes scar. These scars are not desired because they alter the aesthetics of the body, for example, a professional model, when undergoing gallbladder video surgery, may evolve with unwanted scars from video surgery, which may require restorative plastic surgeries. The possibility of performing a surgery without scarring is a major evolution in surgery.

Therefore, what is clearly needed is a surgical device that performs surgery with minimal tissue trauma that is adapted to access the surgical site by a trocar having a plurality of cannulas or by a conventional trocar, and a method of use which solves the problems mentioned above.

### SUMMARY OF THE INVENTION

In one embodiment of the invention, a video-surgical instrument, comprising: (a) a plurality of elongated rods, each including a proximal end and a distal end, (b) a holder, (c) a functional element, and (d) a fairing, wherein each said proximal end is connectable to said holder, wherein each said distal end is connectable to said functional element, wherein said fairing removably covers said plurality of elongated rods, is provided.

Also, in another embodiment of the invention, said holder is in connection with said proximal end to transmit at least one of force, power and torque, to said rods.

Also, in another embodiment of the invention, said fairing covers said rods in a sealing manner.

Also, in another embodiment of the invention, said holder is adapted to interchangeably connect to a plurality of an external apparatus, to transmit at least one of force, power and torque, to at least one said rods.

Also, in another embodiment of the invention, at least one of said rods includes at least one lumen.

Also, in another embodiment of the invention, each said distal end further comprises a piercing tip. Also, in another embodiment, the invention is adapted to cause minimal trauma to the tissue in order to prevent scar.

Also, in another embodiment, the invention further comprising at least one protector guide.

Also, in another embodiment of the invention, said holder further comprises at least one interconnectable modular part.

Also, in another embodiment of the invention, said holder is connectable to at least one external apparatus for transmission of at least one of force, power and torque, to at least one said elongated rods, wherein at least one of said elongated rods is a transmitter of at least one of, force, power and torque, for said functional element.

Also, in another embodiment of the invention, said holder is operatively connected to at least one of said rods and at least one of said rods is operatively connected to at last one said functional element.

Also, in another embodiment of the invention, at least one of said rods is interchangeable with other rods.

Also, in another embodiment of the invention, said functional element is made to perform a surgical operation.

Also, in another embodiment, the invention further comprising electrical connections.

Also, in another embodiment of the invention, said functional element is adapted to interchangeably connect to another functional element inside said surgical site.

Also, in another embodiment, the invention further comprising at least one modular part.

Also, in another embodiment, the invention is adapted to be robotic operating.

Also, in another embodiment, the invention further comprising at least one articulation joint.

Also, in another embodiment of the invention, said functional element is rotatable around one or more axes.

Also, in another embodiment of the invention, said functional element include a camera heard.

Also, in another embodiment of the invention, said instrument is an ultrasonic surgical instrument.

Also, in another embodiment of the invention, said instrument is a surgical stapler.

In another aspect of the invention, a method for performing a surgery, comprising the steps of, (a) inserting a fairing to cover a plurality of elongated rods of a video-surgery instrument, (b) inserting said video-surgery instrument in a surgical site through a conventional trocar, (c) using said video-surgery instrument to perform a surgery, is provided. Also, in another aspect of the invention, said method further comprising interchanging a functional element of said video-surgical instrument to another functional element inside said surgical site, is provided.

In another aspect of the invention, a method of performing a surgery, comprising the steps of, (a) removing a fairing to uncover a plurality of elongated rods of a video-surgery instrument, (b) inserting each said plurality of elongated rods in a surgical site through a trocar comprising a plurality of cannulas, and (c) using said instrument to perform a surgery, is provided. Also, in another aspect of the invention, said method further comprising interchanging at least one said rods with another said rod, is provided. Also, in another aspect of the invention, said method further comprising connecting a functional element to said plurality of elongated rods distal end, is provided. Also, in another aspect of the invention, said method further comprising connecting an external apparatus to said plurality of rods proximal end, is provided. Also, in another aspect of the invention, said method further comprising interchanging said functional element to another said functional element inside said surgical site, is provided.

### BRIEF DESCRIPTION OF THE DRAWINGS

FIG. 1 is a perspective view of a free scar instrument 41, according to another embodiment of the invention.

FIG. 2 is a perspective view of the free scar instrument 41 covered by a fairing 55 according to another embodiment of the invention.

FIG. 3 is a perspective view of the free scar instrument 41 ready to be covered by the fairing 55 according to another embodiment of the invention.

FIG. 4 is a perspective view of the proximal portion 55A of the faring 55 and of the distal portion 3B of the free scar instrument 41, according to another embodiment of the invention.

FIG. 5 is a cross sectional view of the invention showing the direction in which the firing 55 slides longitudinally to cover the plurality of rods 3, according to another embodiment of the invention.

FIG. 6 is a cross sectional view of the invention with the firing 55 covering the plurality of rods 3 in a sealing manner, according to another embodiment of the invention.

FIG. 7 is a perspective view of another embodiment of the invention, including: five elongated rod 3, according to another embodiment of the invention.

FIG. 8 is a perspective view of the elongated rod 3 and the functional element 1, according to another embodiment of the invention.

FIG. 9 is a perspective view of the functional element 1 within the surgical site 26, according to another embodiment of the invention.

FIG. 10 is a perspective view of the invention being used through a conventional trocar 49, according to another embodiment of the invention.

FIG. 11 is a perspective view of the invention showing a method of interchanging the functional element 1 of a video-surgical instrument to another functional element 1 inside the surgical site 26, according to another embodiment of the invention.

FIG. 12 is a lateral view of the invention for manual use according to another embodiment of the invention.

FIG. 13 is a perspective view of the invention, including the protector guide 7, according to another embodiment of the invention.

FIG. 14 is a perspective view of the invention including the trocar 5 and the protector guide 7, according to another embodiment of the invention.

FIG. 15 is a perspective view of the proximal end 3A, the holder 4 and the handle 27E, according to another embodiment of the invention.

FIG. 16 is a perspective view of the proximal end 3A, the holder 4 and the external apparatus 27, according to another embodiment of the invention.

FIG. 17 is a perspective view of a portion of the rod 3, according to another embodiment of the invention.

FIG. 18 is a perspective view of a portion of the command rod 3C, according to another embodiment of the invention.

FIG. 19 is cross sectional view of a portion of the rod 3, according to another embodiment of the invention.

FIG. 20 is a cross-sectional view of a portion of the rods 3 in a rectangular shape, in accordance with another embodiment of the invention.

FIG. 21 is a cross-sectional view of a portion of the rod 3, according to another embodiment of the invention.

FIG. 22 is across sectional view of the rod 3, according to another embodiment of the invention.

FIG. 23 is a cross sectional view of the rod 3, according to another embodiment of the invention.

FIG. 24 is a cross sectional view of the rod 3, according to another embodiment of the invention.

FIG. 25 is a cross sectional view of the rod 3, according to another embodiment of the invention.

FIG. 26 is a perspective view of the invention showing the quick connection 45, according to another embodiment of the invention.

FIG. 27 is a perspective view of another embodiment of the invention configurated to connect to a robotic arm 27D, according to another embodiment of the invention.

FIG. 28 is a side lateral cross-sectional view of the invention adapted to be robotic operating, according to another embodiment of the invention.

FIG. 29 is the lateral view of the invention, including the ultrasonic scissors, according to another embodiment of the invention.

FIG. 30 is a cross-sectional view of another embodiment of the invention, including an ultrasonic scissors, according to another embodiment of the invention.

FIG. 31 is the lateral view of another embodiment of the invention including the ultrasonic scissors 27F, according to another embodiment of the invention.

FIG. 32 and FIG. 33 are perspective views of other embodiments of the invention, in which the functional element 1 is articulable rotating, opening and closing its jaws 10A and jaws 10B, according to another embodiment of the invention.

FIG. 34 is a perspective view of the tool 8, the adapter 2 and the connector 37 and the distal end 3B, according to another embodiment of the invention.

FIG. 35 is a perspective view of the invention, including the functional element 1, the tool 8, an adapter 2, a connector 37 and seven elongate rods 3 which are sectioned to be viewed as the distal end 3B, according to another embodiment of the invention.

FIG. 36 and FIG. 37 are perspective views of the distal end 3B and the protector guide 7, according to another embodiment of the invention.

FIG. 38 and FIG. 39 are perspectives views of a link 18, connection according to another embodiment of the invention.

FIG. 40 is a perspective view of the functional element 1, according to another embodiment of the invention.

FIG. 41 This is a perspective view of a portion of the functional element 1 directly connected to the distal end 3B, according to another embodiment of the invention.

FIG. 42 is another embodiment of the invention that uses the link 18 to connect the rod 3 to the adapter 2.

FIG. 43 is an exploded perspective view of the functional element 1, according to another embodiment of the invention.

FIG. 44 is another view of the functional element 1 connected to an adapter 2, according to another embodiment of the invention.

FIG. 45 It is a trans sectional view of the adapter 2 with four cart 23, according to another embodiment of the invention.

FIG. 46 is a perspective view of the cart 23 for quick connection 45, according to another embodiment of the invention.

FIG. 47 and FIG. 48 are a perspective view of the quick connection 45, according to another embodiment of the invention.

FIG. 49 is a perspective view of the quick connection 45, according to another view of the invention, according to another embodiment of the invention.

FIG. 50 is a lateral view of the distal end 3B including the piercing tip 6, according to another embodiment of the invention.

FIG. 51 is a lateral view of the distal end 3B of the rod 3, including four trocars 5 and four cannulas 5A, according to another embodiment of the invention.

FIG. 52 is a lateral view of the distal end 3B including a plurality of trocars 5 and a protector guide 7, according to another embodiment of the invention.

FIG. 53 is a perspective view of the video camera 27C, according to another embodiment of the invention.

FIG. 54 is a perspective view of the functional element 1, including a camera heard 14, according to another embodiment of the invention.

### DETAILED DESCRIPTION OF THE PREFERRED EMBODIMENTS

The inventor provides a surgical device that performs surgery with minimal tissue trauma that is adapted to access the surgical site by a trocar having a plurality of cannulas or by a conventional trocar, and a method of use which solves the problems mentioned above. The invention is described in enabling detail in the following examples, which may represent more than one embodiment of the invention, together with the accompanying drawings in which like numerals represent similar components. Additionally, the structures described herein can be embodied as integrated components or as separate components.

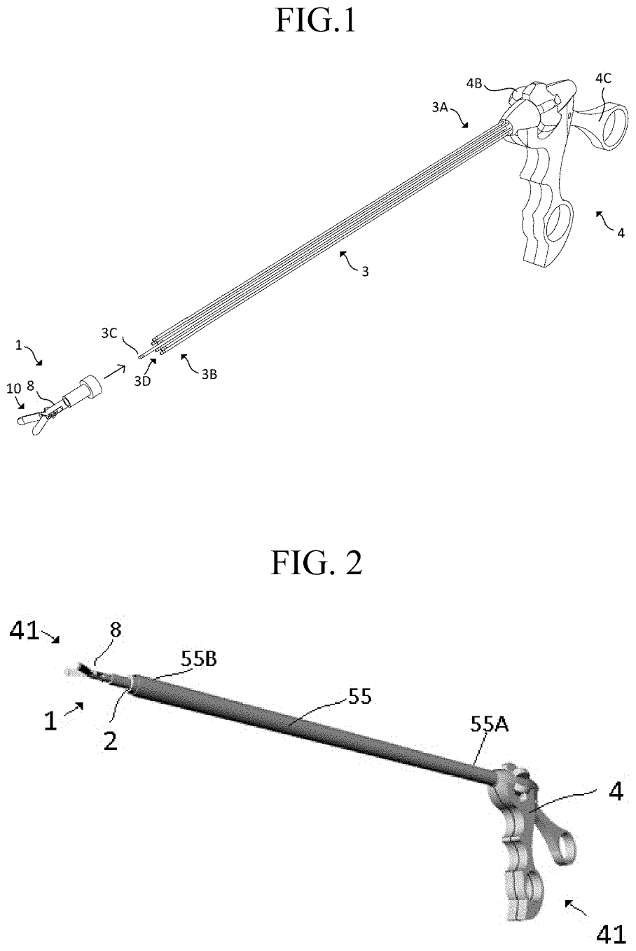

The preferred embodiments according to the invention are shown in FIG. 1-54

FIG. 1 is a perspective view of a free scar instrument 41 including: a plurality of rods 3, a proximal end 3A and a distal end 3B, a holder 4 connected to the proximal end 3A and a functional element 1 is detachably connected to the distal end 3B, according to one embodiment of the invention. In one characteristic of this embodiment, the plurality of rods 3 are adapted to be covered by a fairing 55. In another characteristic of this embodiment, the holder 4 includes a spinner 4B and a lever 4C. In other characteristic of this embodiment, the holder 4 is not modular, and the holder 4 is in connection to the proximal end 3A in a not disconnecting manner. In another characteristic of this embodiment, the functional element 1 is completely detachable from the distal end 3B, the arrow shows the direction in which the functional element 1 connects to the distal end 3B. In another characteristic of this embodiment, the holder 4 is configurated to be manually operating. In another characteristic of this embodiment, the holder 4 is adapted to use the command rods 3C to control the functional element 1. One characteristic of this embodiment is that one command rod 3C is operatively connected to the functional element 1. In other characteristic of this embodiment, the command rod 3C is a transmission member, at least one of, force, power and torque to the functional element 1.

In another characteristic of this embodiment, six support rods 3D are connecting the functional element 1 with the support function.

In another characteristic of this embodiment, the functional element 1 is rotatable around its own axis. In another characteristic of this embodiment, the spinner 4B is operably connected to the rod 3C. In another characteristic of this embodiment, manually rotating the spinner 4B causes the rotation of the support rod 3D that rotates the functional element 1.

In other characteristic of this embodiment, the functional element 1 is made to perform a surgical operation. In another characteristic of this embodiment, the functional element 1 further includes a tool 8. In other characteristic of this embodiment, a tool 8 includes a pair of cooperating jaws 10 made to perform a surgical operation. In other characteristic of this embodiment, the lever 4C is operably connected to the command rod 3C. In other characteristic of this embodiment, the longitudinal movement of the lever 4C is transmitted to the command rod 3C. In other characteristic of this embodiment, the longitudinal movement of the command rod 3C is transmitted to the functional element 1. In other characteristic of this embodiment, the longitudinal movement of the command rod 3C opens and closes the jaws 10 of the functional element 1. In another characteristic of this embodiment, the holder 4 is configurated to be manually operating.

In another characteristic of this embodiment, the distal end 3B is adapted to interchangeably connect to a plurality of the functional elements 1. In another characteristic of this embodiment the functional element 1 is adapted to interchangeably connect to another functional element 1.

In one characteristic of some embodiments, the functional element 1 includes the tool 8 to perform a surgery operation. In some embodiment the tool 8 is an ultrasonic scissors; In some embodiment the tool 8 is a camera heard 14; in some embodiment the tool 8 is a tweezer; in some embodiment the tool 8 is a suture device; in some embodiments the tool 8 is scissors; in some embodiments the tool 8 is a surgical stapler; in some embodiments the tool 8 includes a stapler loading; in some embodiments the tool 8 is a clamp; in some embodiments the tool 8 is a linkage mechanism; in some embodiments the tool 8 includes an electrical device, in some embodiments the tool 8 includes a motor; in some embodiments the tool 8 includes a solenoid; in some embodiments the tool 8 includes an electric scalpel; in some embodiments the functional element 1 includes an ultrasonic scissors; in some embodiments the functional element 1 includes an ultrasonic device; in some embodiments the functional element 1 includes a retractor; in some embodiments the functional element 1 includes a vacuum cleaner; in some embodiments the functional element 1 includes an optics; in some embodiments the functional element 1 includes a suture device; in some embodiments the functional element 1 includes a needle holder 4: in some embodiments the functional element 1 includes a Laser; in some embodiments the functional element 1 includes a bag; in some embodiments the functional element 1 includes a robot; in some embodiments the functional element 1 includes servomechanism; in some embodiments the functional element 1 includes a motor; in some embodiments the functional element 1 includes a servo-motor, in some embodiments the functional element 1 includes a surgical stapler, but is not limited to them; in some embodiments the invention further comprising electrical connections.

FIG. 2 is a perspective view of the free scar instrument 41 covered by a fairing 55 according to another embodiment of the invention. This embodiment including: a plurality of rods 3, each including a proximal end 3A and a distal end 3B; a holder 4; a functional element 1 and (d) a fairing 55; wherein each rods 3 proximal end 3A is connectable to the holder 4; wherein each said rods 3 distal end 3B is connectable to the functional element 1; wherein said fairing 55 removably covers said plurality of rods 3. In this embodiment the holder 4 is in connection with said proximal end 3A to transmit at least one of force, power and torque, to said rods 3. In one characteristic of this embodiment, the free scar instrument 41 when coated with the fairing 55 form a single set and acts as a conventional video-surgery instrument, it is adapted to be inserted and used in a conventional trocar 49 without escape of the contents out of a surgical site 26. In one characteristic of this embodiment, the fairing 55 is introduced longitudinally by the distal end 3B of the free scar instrument 41 and slides longitudinally to completely cover the plurality of rods 3.

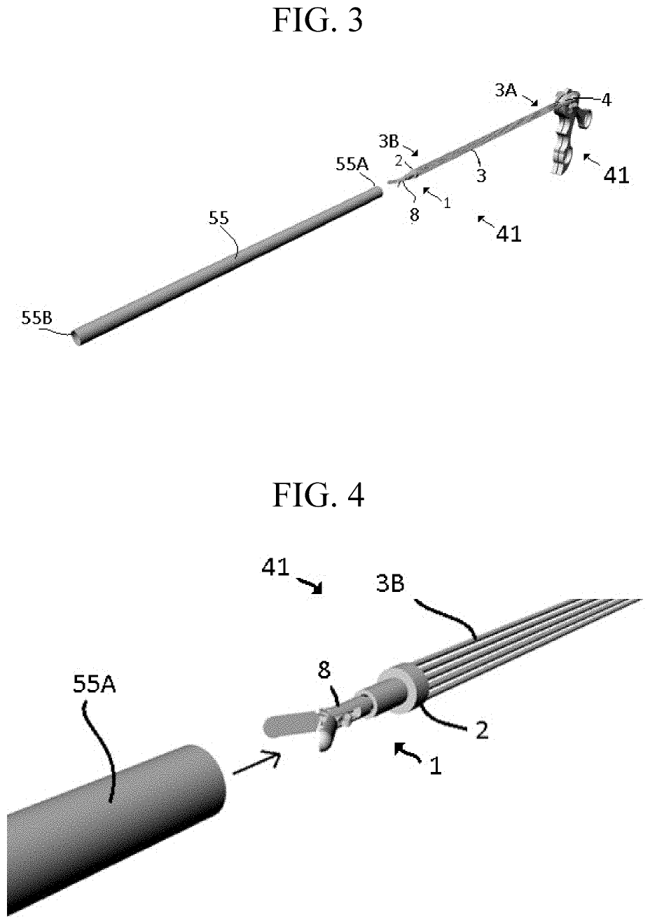

FIG. 3 is a perspective view of the free scar instrument 41 ready to be covered by the fairing 55, according to another embodiment of the invention. In one characteristic of this embodiment, the fairing 55 is ready to slide longitudinally to cover the plurality of elongate rods 3. In one characteristic of this embodiment, the video surgical instrument including a plurality of rods 3 when coated with the fairing 55 form a single set and acts as a conventional video-surgery instrument, it is adapted to be inserted in a conventional trocar 49. In one characteristic of this embodiment, the faring 55 is introduced longitudinally by the distal end 3B of the instrument and slid ably placed to completely cover the plurality of rods. In one characteristic of this embodiment, the fairing 55 covers said rods 3 in a sealing manner.

In one characteristic of this embodiment, the video surgical instrument including a plurality of rods 3 when is covered by the fairing 55, externally closely resembles a conventional video-surgery instrument, and is adapted to be used normally as the conventional video-surgery instrument.

FIG. 4 is a perspective view of the proximal portion 55A of the faring 55 and of the distal portion 3B of the free scar instrument 41, according to another embodiment of the invention. In one characteristic of this embodiment, the functional element 1, and the plurality of rods 3 are connected and are not covered by faring 55. The arrow shows the direction of placement of the faring 55 in the free scar instrument 41.

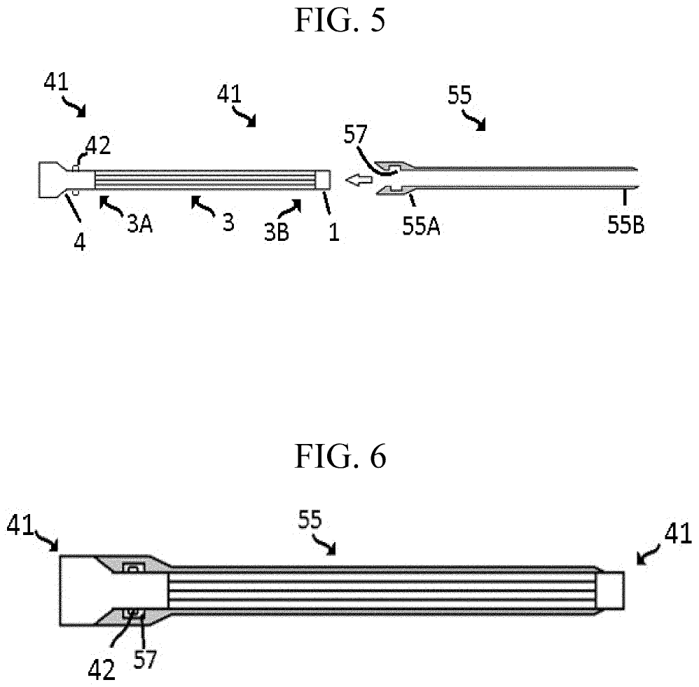

FIG. 5 is a cross sectional view of the invention showing the direction in which the firing 55 slides longitudinally to cover the plurality of rods 3, according to another embodiment of the invention. In one characteristic of this embodiment, the fitting 57 connect to the sealing 42 in a way to avoid scraping of gas and liquid from the surgical site 26 when the invention is inserted in a conventional trocar 49.

FIG. 6 is a cross sectional view of the invention with the firing 55 covering the plurality of rods 3 in a sealing manner, according to another embodiment of the invention. There is a sealing 42 on the proximal part 55A of the fairing 55 that prevents the escape of the contents of the surgical site 26. In some embodiments the sealing 42 is in the distal part 55B of the faring 55, in some embodiment the sealing 42 is in the functional element 1, in some embodiment the sealing 42 is in the holder 4, but the position of the sealing 42 is not limited to them. In some embodiment there is no sealing 42. In some embodiment there are more than one sealing 42. In some embodiments, the sealing 42 engages sealing on the adapter 2.

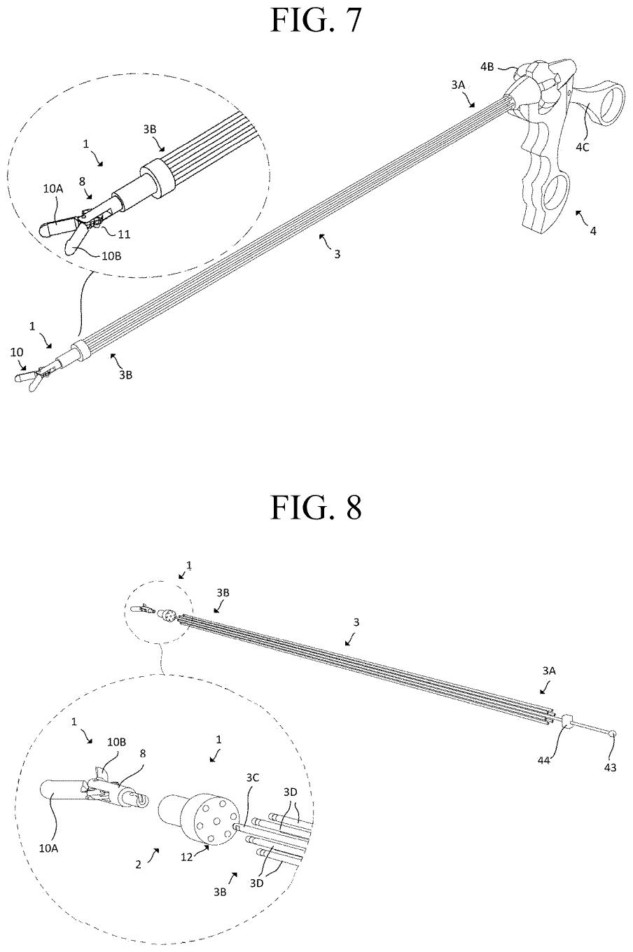

FIG. 7 is a perspective view of another embodiment of the invention, including: five rod 3, according to another embodiment of the invention. In this embodiment, the rod 3 including the proximal end 3A and the distal end 3B, the holder 4 connected to the proximal end 3A, and the functional element 1 detachably connected to the distal end 3B. In one characteristics of this embodiment, the distal end 3B and the functional element 1 are seen in the detail of the FIG. 7.

In another characteristic of this embodiment, the holder 4 is not modular, and the holder 4 is connected to the proximal end 3A in a non-disconnecting manner. In another characteristic of this embodiment, the functional element 1 is connected to the distal end 3B.

In another characteristic of the invention, the rod 3 connect the holder 4 to the functional element 1. In another characteristic of this embodiment, the holder 4 is adapted to actuate the functional element 1 via the command rod 3C. In another characteristic of this embodiment, the holder 4 includes the spinner 4B and the lever 4C. In one characteristics of this embodiment, the spinner 4B is attached to the transitional command rod 3C (it is not shown in the drawing). In other characteristic of this embodiment, the functional element 1 may be rotated relative to the longitudinal axis from the rotation of the spinner 4B. In another characteristic of this embodiment, the transitional command rod 3C is connected to the functional element 1, the rotation of the transitional command rod 3C causes the functional element 1 to rotate. In another characteristic of this embodiment, the spinner 4B rotates causes the transitional command rod 3C to rotate the functional element 1.

In other characteristic of this embodiment, the functional element 1 comprises a pair of cooperating jaws 10 made to perform a surgical operation. In another characteristic of this embodiment, the functional element 1 comprises an articulation joint 11, to open and close the jaws 10. In other characteristic of this embodiment, the functional element 1 is detachably connected to the command rod 3C.

In one characteristics of this embodiment, the longitudinal movement of the lever 4C, the opening and closing of the lever 4C, causes a longitudinal movement of the command rod 3C this movement opens or closes the jaws 10 of the functional element 1.

In another characteristic of this embodiment, the functional element 1 is adapted to interchangeably connect to another functional element 1. In another characteristic of this embodiment, the functional element 1 may be disconnected from the distal end 3B and another functional element 1 may be connected to the distal end 3B.

In another characteristic of this embodiment, the functional element 1 includes a gripper tool 8. In another characteristic of this embodiment, the tool 8 includes two cooperative jaws 10. In other characteristic of this embodiment, the command rod 3C connects the functional element 1 to activate a mechanism to open and close the jaws 10.

In other characteristic of this embodiment, that command rod 3C is a transmission member of force to open and close the jaws 10 of the gripper. Also, the same command rod 3C is a transmission member of torque to rotate the functional element 1 in relation to the rods 3 longitudinal direction.

In one characteristic of some embodiments, at least one the rods 3 is a transmission member, at least one of, force, power and torque. In one characteristic of some embodiments, other command rods 3C may function to activate a mechanism in the functional element 1 from the holder 4, for example: a button on the holder 4 that disconnects the functional element 1.

In some embodiments, the holder 4 is adapted to use the command rod 3C to control the functional element 1.

FIG. 8 is a perspective view of the rod 3 and the functional element 1, according to another embodiment of the invention.

In this example, the holder 4 is not displayed, the functional element 1 is disconnected from the distal end 3B. In other characteristic of this embodiment, the command rod 3C is longer than the support rod 3D to connect the base 44 to the spinner 4B. In another characteristic of this embodiment, the base 44 of the command rod 3C connects to the spinner 4B to rotate the command rod 3C and the command rod 3C rotates the functional element 1.

In another characteristic of this embodiment, a boll 43 connects to a lever 4C to act on the functional element 1. In this example, the functional element 1 includes the tool 8 and the adapter 2. In this example, the command rod 3C extends beyond the proximal end 3A. In other characteristic of this embodiment, the command rod 3C is adapted to connect to the holder 4. In this example, the command rod 3C is ready to connect to the pit 2B and the support rods 3D are ready to connect to the hole 2A.

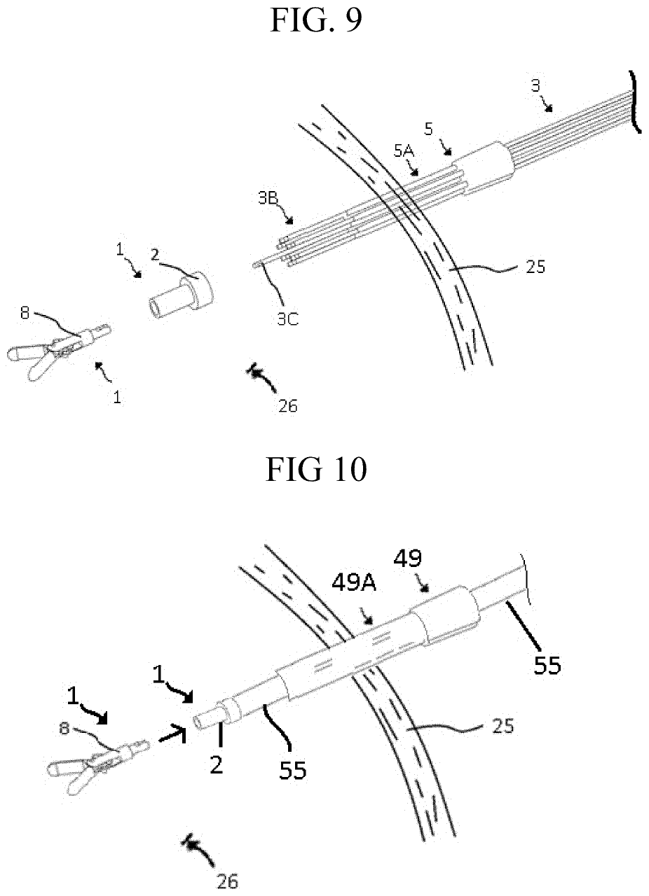

FIG. 9 is a perspective view of the functional element 1 within the surgical site 26, according to another embodiment of the invention. In this example the plurality of rods 3 are inserted into the surgical site 26 through a plurality of perforations in a tissue 25. In another characteristic of this embodiment, one part of the plurality of rod 3 is seen, the plurality of rod 3 is perforating the tissue 25 through the plurality cannulas trocar 5, the proximal end 3A is inside the surgical site 26.

In this example, the functional element 1 is ready to be connected to the distal end 3B inside the surgical site 26. In another characteristic of this embodiment, the functional element 1 is modular, including the adapter 2 and the tool 8.

In this example, the adapter 2 is disconnected from the distal end 3B, the tool 8 is disconnected from the adapter 2. In other characteristic of this embodiment, the rod 3 passes through a cannulas 5A of the trocar 5 to access the surgical site 26. In other characteristic of this embodiment, the trocar 5 has the plurality of cannulas 5A which are inserted into a surgical site 26 through a plurality of perforations in the tissue 25 without causing scarring. In other characteristic of this embodiment, the trocar 5 is adapted to perforate the tissue 25 without scarring. In another characteristic of this embodiment, the cannulas 5A have an external diameter sufficiently thin to avoid causing scars on the tissue 25. In this example, the rod 3 passes through the trocar 5, through a passageway 28 of the cannulas 5A. In this example, the distal end 3B is free to connect to the adapter 2, the adapter 2 is ready to be inserted into the distal end 3B of the rod 3, and the tool 8 is ready to be inserted into the adapter 2. In other characteristic of this embodiment, the invention is interchangeably on the functional element 1 with another functional element 1. In another characteristic of this embodiment, the tool 8 is adapted to be disconnected from the adapter 2 and replaced by another tool 8. In another characteristic of this embodiment, the adapter 2 is adapted to be disconnected from the distal end 3B and replaced by another adapter 2 In one characteristic of some embodiments, the functional element 1 is modular, including a plurality of detachably connected parts. In one characteristic of some embodiments, the trocar 5 is part of the invention. In some embodiments, the invention further comprises at least one trocar 5.

In one characteristic of this embodiment is the method of performing a surgery, comprising the steps of: removing the fairing 55 to uncover a plurality of rods 3 of the free scar instrument 41; inserting each said plurality of rods 3 in a surgical site 26 through one cannula 5A of the trocar 5 comprising a plurality of cannulas 5A; and using the free scar instrument 41 to perform a surgery, is provided. In one characteristic of this embodiment, the method further comprising interchanging at least one the rods 3 with another the rod 3. In one characteristic of this embodiment, the method further comprising connecting a functional element 1 to the plurality of rods 3 distal end 3B; the method further comprising connecting an external apparatus 27 to said plurality of rods 3 proximal end 3A. In one characteristic of this embodiment, the method further comprising interchanging said functional element 1 to another said functional element 1 inside the surgical site 26.

FIG. 10 is a perspective view of the invention being used through a conventional trocar 49. In one characteristic of this embodiment, the method of performing a surgery, comprising the steps of inserting a fairing 55 to cover a plurality of rods 3 of a video-surgery instrument; inserting said instrument in a surgical site through a conventional trocar 49; using said instrument to perform a surgery, is provided. The conventional trocar 49 has a single cannula 49A.

In this embodiment the video surgical instrument is the free scar instrument 41 with the rods 3 covered by the fairing 55.

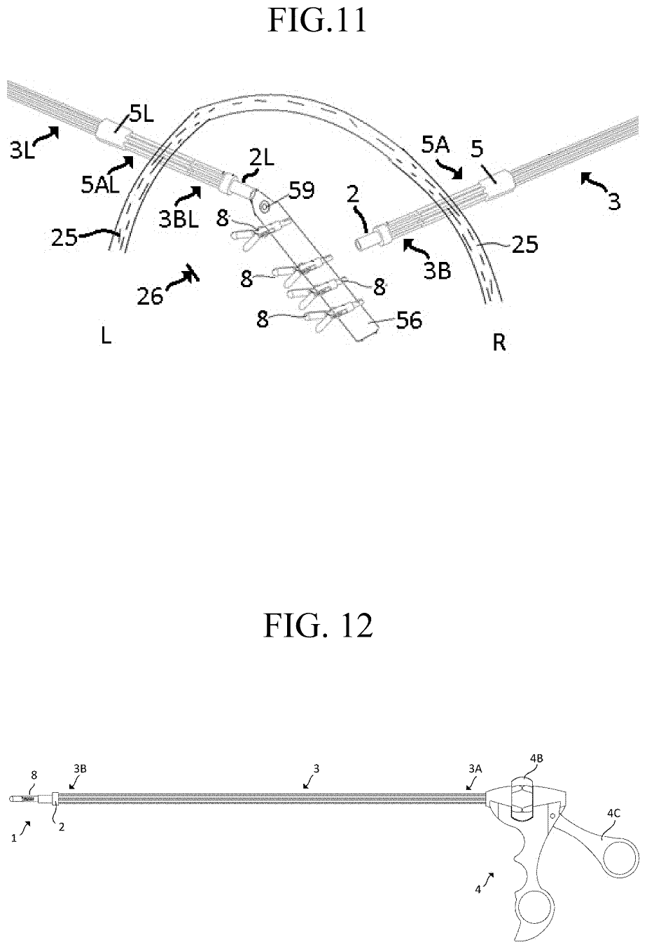

FIG. 11 is a perspective view of the invention showing a method of interchanging the functional element 1 of a video-surgical instrument to another functional element 1 inside the surgical site 26, according to another embodiment of the invention. Two free scar instruments 41 with removable tool 8 are seen, one on the right side and the other on the left side, one inserted in plurality cannulas trocar 5 and the other in a plurality cannulas trocar 5L. In one characteristic of this embodiment, the plurality cannulas 5AL of the plurality cannulas trocar 5L is crossing the tissue 25. In one characteristic of this embodiment, the plurality cannulas 5A of the plurality cannulas trocar 5 is also crossing the tissue 25 in another site. In one characteristic of this embodiment, the two instruments are inserted in the surgical site 26. On the left side, four tools 8 are connected to the tool 8 that is a toolbox 56. In one characteristic of this embodiment, the toolbox 56 is connected to the adaptor 2L. In one characteristic of this embodiment, the toolbox 56 is connected to four tools 8 which are available to be chosen and connected to the adapter 2.

FIG. 12 is a lateral view of the invention for manual use according to another embodiment of the invention.

In another characteristic of this embodiment, the functional element 1 is connected to the distal end 3B. In another characteristic of this embodiment, the holder 4 includes the spinner 4B and the lever 4C. In other characteristic of this embodiment, the holder 4 is not modular and the holder 4 is connected to the proximal end 3A in a non-disconnecting manner.

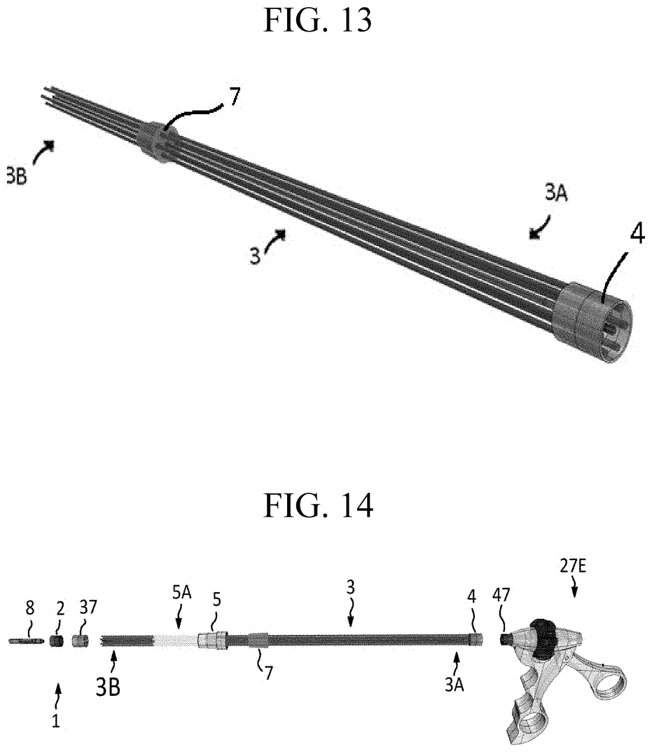

FIG. 13 is a perspective view of the invention, including a protector guide 7, according to another embodiment of the invention. This embodiment is another example of a surgical device for insertion into a surgical site 26 through a plurality of perforations in a tissue 25, including: a plurality of rods 3, the rods 3 including proximal ends 3A and distal ends 3B, and the holder 4 connected to the proximal end 3A, further including one the protector guide 7.

In another characteristic, this embodiment includes seven rods 3. In another characteristic of this embodiment, the holder 4 is connectedly fixed to the proximal end 3A.

In another characteristic of this embodiment, the holder 4 is adapted to interchangeably connect to a plurality of external apparatus 27 for the transmission of, at least one of, force, power and torque, for the rod 3. In another characteristic of this embodiment, the holder 4 is adapted to functionally connect the plurality of external apparatus 27. In another characteristic of this embodiment, the rod 3 is adapted to work as a support rod 3D or as a command rod 3C according on the external apparatus 27 to connect. In another characteristic of this embodiment, it has six support rods 3D and one command rod 3C. In one characteristic of some embodiments, the holder 4 is adapted to slide on the rod 3 along the length of the rod 3 or by a segment of the rod 3. In one characteristic of some embodiments, the rods 3 are solid. In one characteristic of some embodiments, the rods 3 have the lumen 9. In one characteristic of some embodiments, through a lumen 9 of the rods 3, passes the nuclear rod 3E. In one characteristics of some embodiment, the nuclear rods 3E are structures for: power transmission, torque transmission and force transmission, for example: electrical connection, cable, rods, hydraulic structures, but is not limited to them.

In another characteristic of this embodiment, the distal end 3B is adapted to interchangeably connect to a plurality of the functional elements 1. In one characteristics of some embodiment, the functional element 1 is a surgical stapler to make an anastomosis.

In another characteristic of this embodiment, at least one of the rods 3 is interchangeable with another rod 3. In another characteristic of this embodiment, each rod 3 has a specific function, for example: during the procedure, the mechanical command rod 3C is adapted to be replaced by the hydraulic command rod 3C the command rod 3C is adapted to be removed and replaced by another command rod 3C. In other characteristic of this embodiment, the command rod 3C has a hydraulic mechanism, or a mechanical mechanism, or an electrical mechanism, but it is not limited to them. Some rods 3 are adapted to be replaced and connected to different external apparatus 27 and to different functional elements 1. In other characteristic of this embodiment, there is an auxiliary device for the replacement. In other characteristic of this embodiment, during a procedure, the rod 3 is adapted to be removed from the plurality of rods 3 and replaced with another rod 3.

In one characteristic of some embodiments, there is a mechanism for releasing the holder 4, the holder 4 is adapted to be detached and replaced. In one characteristic of some embodiments, the holder 4 is adapted to move longitudinally on the rod 3.

FIG. 14 is a perspective view of the invention including the trocar 5 and the protector guide 7, according to another embodiment of the invention.

This embodiment is modular, the handler 27 E is connectable to the holder 4, and the functional element 1 is connectable to the distal end 3B. In another characteristic of this embodiment, the functional element 1 is modular including a connector 37, the adapter 2, and the tool 8. This embodiment including eight rods 3, the eight rods 3 including proximal ends 3A and distal ends 3B and the trocar 5. In another characteristic, this embodiment further includes a modular functional element 1 detachably connected to the distal end 3B. In another characteristic of this embodiment, the functional element 1 is interchangeable with another functional element 1 and other assemblies and surgical instruments, the functional element 1 is adapted to be disconnected and connected to another type of functional element 1.

In another characteristic of this embodiment, the trocar 5 includes eight cannulas 5A. In other characteristic of this embodiment, the holder 4 is adapted to connect to an external apparatus 27 for the transmission of, at least one of, force, power and torque, for the functional element 1. In other characteristic of this embodiment, the external apparatus 27 is a handler 27E adapted for manual operation. In other characteristic of this embodiment, the modular functional element 1 is made to perform a surgical operation.

In another characteristic of this embodiment, the holder 4 is connectable to the external apparatus 27.

In another characteristic of some embodiment, the holder 4 is completely detachable from the handler 27E. In another characteristic of this embodiment, the handler 27E is operatively connected to the holder 4. In another characteristic of some embodiment, the handler 27E is configurated to be manually operating.

In another characteristic of some embodiment, a protector guide 7 is detachably connectable to the trocar 5. In one characteristic of some embodiments, the holder 4 is modular, including detachably connected parts.

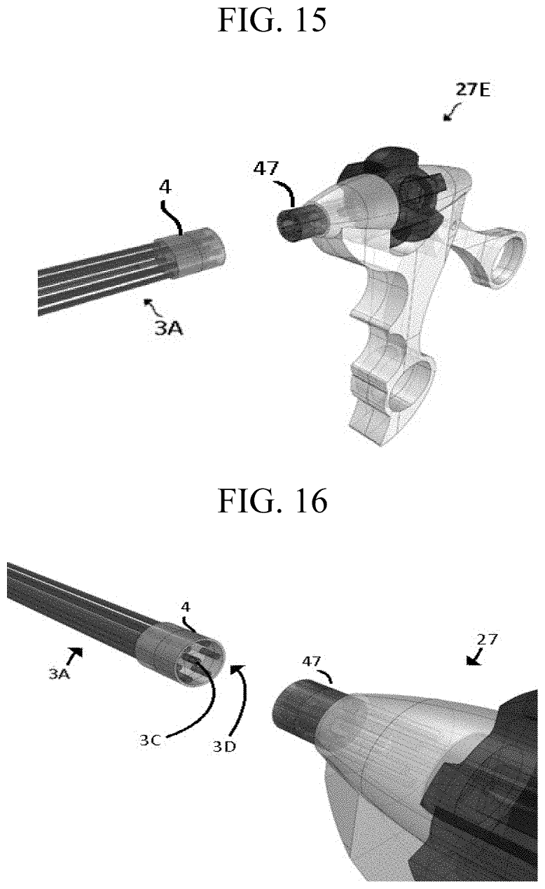

FIG. 15 is a perspective view of the proximal end 3A, the holder 4 and the handle 27E, according to another embodiment of the invention.

In another characteristic of this embodiment, the holder 4 is adapted to connect to at least one external apparatus 27 for the transmission of, at least one of, force, power and torque, for the functional element 1.

In another characteristic of this embodiment, the proximal 3A is adapted to interchangeably connect to a plurality of the external apparatus 27.

In another characteristic of this embodiment, the holder 4 is adapted to interchangeably connect to a plurality of the external apparatus 27.

In another characteristic of this embodiment, the holder 4 is operatively connectable to at least one of the rods 3 and at least one of the command rods 3C and is operatively connectable to the functional element 1.

In another characteristic of this embodiment, the rods 3C are functionally connectable to the holder 4 and functionally connectable to the functional element 1.

In another characteristic of some embodiment, at least one of the rods 3 is a transmitter of, at least one of, force, power and torque, for the functional element 1, the command rod 3C. In another characteristic of some embodiment, the holder 4 is adapted to interchangeably connect to a plurality of external apparatus 27 for the transmission of, at least one of, force, power and torque, for the command rod 3C and for the functional element 1.

In another characteristic of some embodiment, the external device 27 further includes at least one motor. In another characteristic of some embodiment, the external device includes one battery. In another characteristic of some embodiment, it is connectable to the external apparatus 27 including a surgical stapler manipulator 27 G. In another characteristic of some embodiment, is connectable to the external apparatus 27 including a light source 27B. In another characteristic of some embodiment, is connectable to the external apparatus 27 including a robotic arm 27D. In another characteristic of some embodiment, is connectable to the external apparatus 27 including an ultrasonic device 27F. In another characteristic of some embodiment, is connectable to the external apparatus 27 including a surgical robot 27H. In another characteristic of some embodiment, is connectable to the external apparatus 27 including a laser 27 J. In another characteristic of this embodiment, is connectable to the external apparatus 27 including a servomotor 27K. In another characteristic of some embodiment, is connectable to the external apparatus 27 including an electric source 27L. In another characteristic of this embodiment, is connectable to the external apparatus 27 including a power source 27M. In another characteristic of some embodiment, is connectable to the external apparatus 27 including an ultrasonic generator 35. In another characteristic of this embodiment, is connectable to the external apparatus 27 including an ultrasonic device but is not limited to them.

In another characteristic of this embodiment, the functional element 1 is made to perform a surgical operation. In another characteristic of this embodiment, the holder 4 is connectable to the proximal end 3A. In another characteristic of this embodiment, the rods 3 are interchangeable with other rods 3. In some embodiments, the surgical stapler manipulator 27 G is adapted to be robotic operating.

In one characteristic of some embodiments, the surgical stapler manipulator 27 G is modular, including detachably connected parts. In one characteristic of some embodiments, the battery is a detachably connected modular part.

In one characteristic of some embodiments, the surgical stapler manipulator 27 G includes a servo motor, a motor or a linear actuator to actuate at least one of the command rods 3C.

In one characteristic of some embodiments, the surgical stapler manipulator 27 G includes at least one battery. In one characteristic of some embodiments, the surgical stapler manipulator 27 G is a supplier member, at least one of, force, power and torque to, at least, one rod 3. In some embodiments the invention includes the external apparatus 27.

FIG. 16 is a perspective view of the proximal end 3A, the holder 4 and the external apparatus 27, according to another embodiment of the invention. In another characteristic of this embodiment, the invention is exchanged in the holder 4 with various types of external apparatus 27, with other surgical instruments and surgical devices. In another characteristic of this embodiment, the surgery may be initiated using an external apparatus 27 configurated for manual use, the holder 4 is adapted to be disconnected from an external apparatus 27 and connected to another external apparatus 27. In another characteristic of this embodiment, the invention is adapted to be connected to a robotic arm 27D, subsequently disconnected of the robotic arm 27D and connected to another robotic arm 27D but is not limited to them.

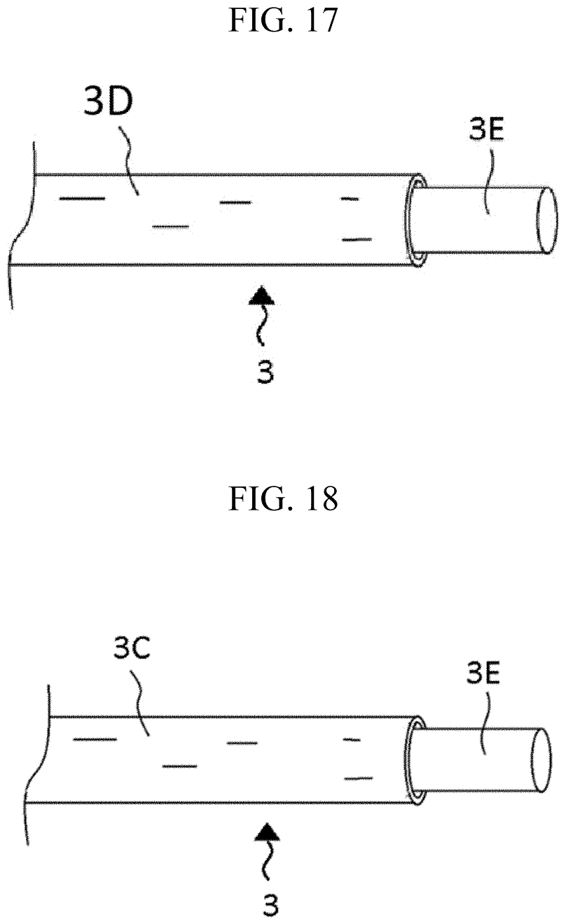

FIG. 17 is a perspective view of a portion of the rod 3, according to another embodiment of the invention.

In other characteristic of this embodiment, the rod 3 includes the support rod 3D and the nuclear rod 3E. In other characteristic of this embodiment, the rod 3 is a tube, the nuclear rod 3E is located within the support rod 3D.

In other characteristic of some embodiments of the invention, the nuclear rod 3E has the same function of the command rod 3C. In one characteristics of this embodiment, the command rod 3C moves longitudinally in relation to the support rod 3D. Also, the command rod 3C rotates with respect to support rod 3D.

FIG. 18 is a perspective view of a portion of the command rod 3C according to another embodiment of the invention.

In other characteristic of this embodiment, the rod 3 includes the command rod 3C and the nuclear rod 3E. Other characteristic of this embodiment of the invention, the nuclear rod 3E is adapted to work as the support rod 3D, and the nuclear rod 3E has a support function. In another characteristic of this embodiment, the command rod 3C moves longitudinally in relation to the nuclear rod 3E. In another characteristic of this embodiment, the command rod 3C moves longitudinally in relation to the nuclear rod 3E. Also, the command rod 3C rotates with respect to the nuclear rod 3E.

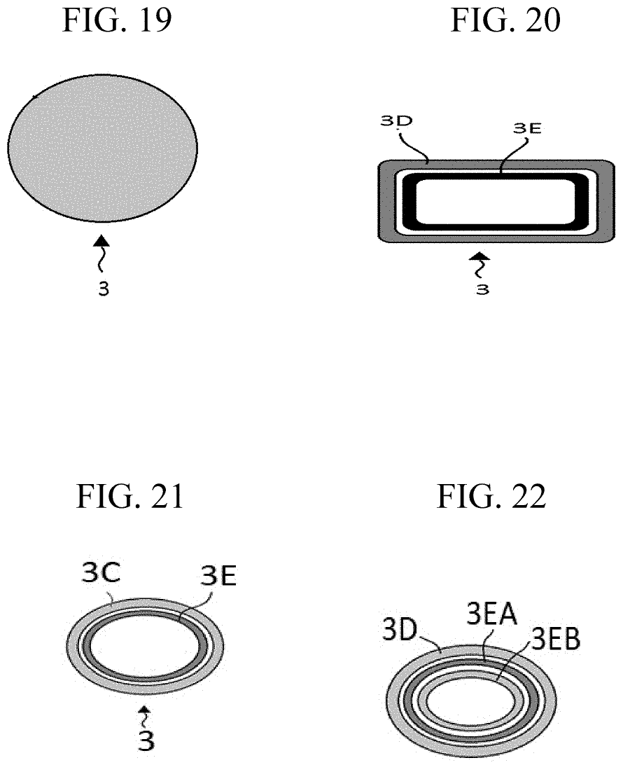

FIG. 19 is cross sectional view of a portion of the rod 3 according to another embodiment of the invention.

In another characteristic of this embodiment, the rod 3 is solid. In other characteristic of this embodiment, the rod 3 is adapted to be used as the command rod 3C or as the support rod 3D. In another characteristic of this embodiment, the rods 3 are the command rods 3C. In another characteristic of this embodiment, the support rod 3D has a support function.

FIG. 20 is a cross-sectional view of a portion of the rods 3 in a rectangular shape, in accordance with another embodiment of the invention.

In other characteristics of this embodiment, the support rod 3D may have the nuclear rod 3E within it. In another characteristic of this embodiment, the nuclear rod 3E is adapted to work as the removable rod 3F. In another characteristic of this embodiment, the nuclear rod 3E is adapted to work as the command rod 3C or as the support rod 3D.

FIG. 21 is a cross-sectional view of a portion of the rod 3 according to another embodiment of the invention.

In another characteristic of this embodiment, the rod 3 includes the command rod 3C and the nuclear rod 3E therein.

In one characteristic of some embodiments, the rod 3 includes the lumen 9 with the nuclear rod 3E therein, and the rod 3 work as the support rod 3D, and the nuclear rod 3E work as the command rod 3C. In other embodiments, the rod 3 includes the lumen 9 with the nuclear rod 3E therein, and the rod 3 work as the command rod 3C and the nuclear rod 3E work as the support rod 3D. In one characteristic of some embodiments, the nuclear rod 3E is a traction cable 16.

FIG. 22 is across sectional view of the rod 3 according to another embodiment of the invention.

In another characteristic of this embodiment, the rod 3 includes the support rod 3D and the nuclear rod 3EA with the other nuclear rod 3EB inside. In another characteristic of this embodiment, the nuclear rod 3EA is inside the other nuclear rod 3EB. In another characteristic of this embodiment, each of the nuclear rod 3E are adapted to work as a command rod 3C. In another characteristic of this embodiment, each nuclear rod 3E is adapted to work as a support rod 3D. In another characteristic of some embodiments of the invention, there are a plurality of nuclear rod 3E. In some embodiments, at least one nuclear rod 3E is the support rod 3D. In another characteristic of some embodiments of the invention, at least one nuclear rod 3E work as the command rod 3C. In one characteristic of some embodiments, the nuclear rod 3E includes an electric wire 53; in some embodiments the nuclear rod 3E includes a liquid for hydraulic action; in some embodiments the nuclear rod 3E includes an optical fiber; in some embodiments the nuclear rod 3E includes a wire; in some embodiments the nuclear rod 3E includes a gas, but is not limited to them.

FIG. 23 is a cross sectional view of the rod 3 according to another embodiment of the invention.

In one characteristic of this embodiment, the rod 3 includes the nuclear rod 3EA and the nuclear rod 3EB with the other nuclear rod 3EC inside. In other characteristics of this embodiment, each nuclear rod 3E command rod 3C.

In another characteristic of this embodiment, the solid nuclear rod 3EB is inside the nuclear rods 3EA. In other characteristics of this embodiment, all the nuclear rod 3E operates independently. In one characteristic of some embodiments, each of the nuclear rod 3E is adapted to rotate relative around to the support rod 3D axis; in one characteristic of some embodiments, each of the 3E rods 3 are adapted to slide lengthwise. In some embodiments, the nuclear rod 3E is a traction cable 16. In some embodiments, the nuclear rod 3E is a wire.

In other characteristics of some embodiments of the invention, the rod 3 includes the support rod 3D and the command rod 3C. In some embodiments, the rod 3 includes at least one support rod 3D. Another characteristic of some embodiments of the invention is that the rod 3 includes at least one command rod 3C.

In other characteristics of some embodiments of the invention, the support rods 3D connects the functional element 1 and to the holder 4 for support function.

In other characteristics of some embodiments of the invention, the command rod 3C connects the holder 4 to the functional element 1 for the transmission of, at least one of: force, power and torque to the functional element 1.

In other characteristics of some embodiments of the invention, the command rod 3C acts on the functional element 1 to make it perform its function. Another characteristic of some embodiments of the invention is that the support rods 3D fixes the functional element 1 to the holder 4.

FIG. 24 is a cross sectional view of the rod 3 according to another embodiment of the invention. In other characteristics of this embodiment, the rod 3 includes three nuclear rod 3E that act independently of one another. In one characteristics of this embodiment, the rod 3 is adapted to move along or rotate. In one characteristic of some embodiments, the nuclear rod 3E is a traction cable 16. In one characteristic of some embodiments, there are two nuclear rods 3E; in some embodiments there are three nuclear rod 3E, but the number of nuclear 3E is not limited to them.

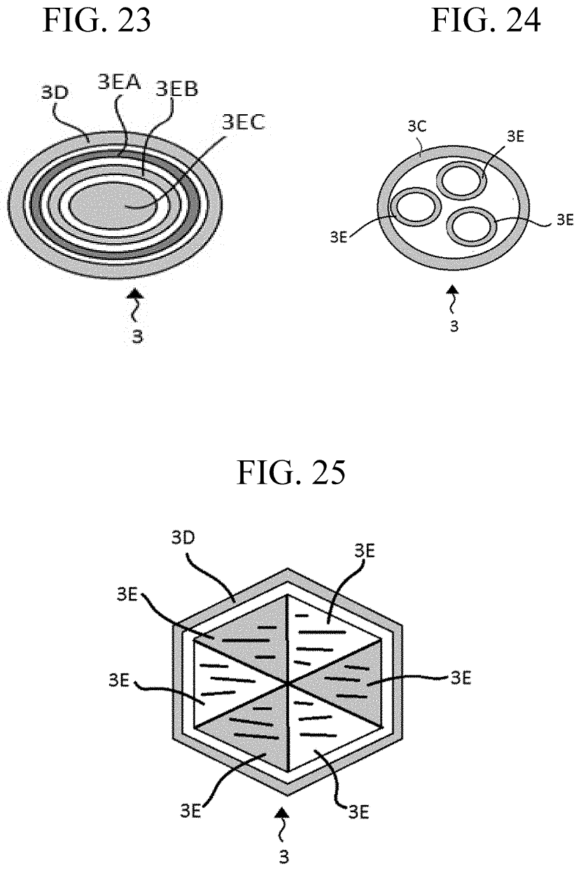

FIG. 25 is a cross sectional view of the rod 3 according to another embodiment of the invention.

In other characteristics of this embodiment, the support rod 3D is hexagonal and includes six nuclear rods 3E in triangular format. In one characteristic of some embodiments, each nuclear rod 3E is adapted to move longitudinally independently one of another. In other characteristic of this embodiment, the command rod 3C is adapted to be connected functionally on the functional element 1 in six different ways, as nuclear rod 3E.

In one characteristic of some embodiments, the support rod 3D connects to the functional element 1 with a support function. In one characteristic of some embodiments, the support rod 3D supports the functional element 1 so the six central nuclear rod 3E are adapted to act on six functional mechanisms of the functional element 1.

In one characteristic of some embodiments, the cross-section shape of the rod 3 is round; in some embodiments, the cross-section shape of the rod 3 is elliptical; in some embodiments, the cross-section shape of the rod 3 is square. In one characteristic of some embodiments, the cross-section shape of the rod 3 is rectangular; in some embodiments, the cross-section shape of the rod 3 is hexagonal, but the cross-sectional shape of the rod 3 is not limited to them. In one characteristic of some embodiments, at least one the rod 3 is electrically insulating. In one characteristic of some embodiments, at least a portion of the rods 3 are electrically insulating, for example: using it with the electric scalpel. In one characteristic of some embodiments, the rod 3 is coated with a layer to facilitate the slippage, and to decrease friction. In one characteristic of some embodiments, the rod 3 are coated with polytetrafluoroethylene.

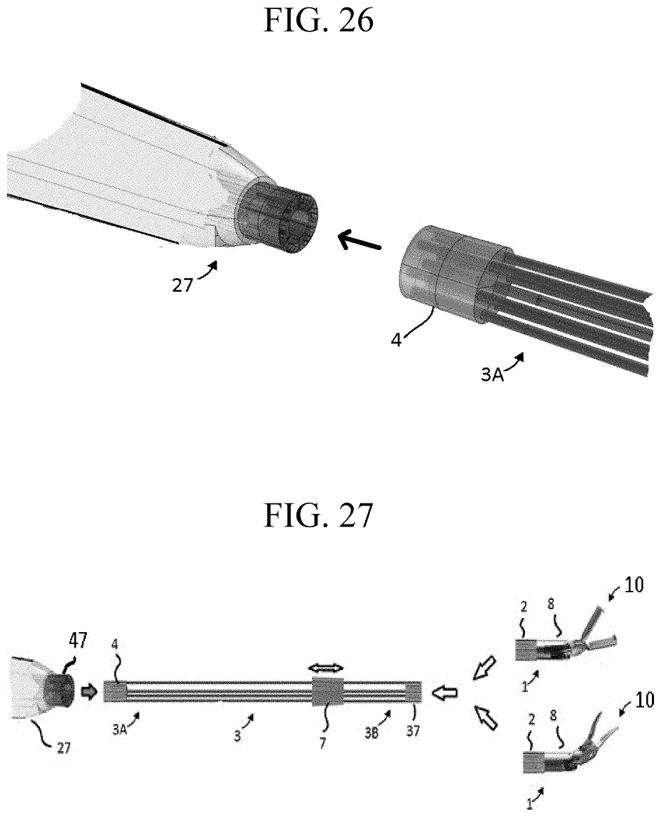

FIG. 26 is a perspective view of the invention showing the quick connection 45 according to another embodiment of the invention.

In other characteristics of this embodiment, the quick connection 45 connects the holder 4 to the external apparatus 27 rapidly. In other characteristics of this embodiment, the holder 4 is connected in the external apparatus 27 in a quick form using the quick connection 45 which allows to connect the holder 4 and all the rods 3 at a single time. In other characteristics of this embodiment, the command rod 3C connects to the external apparatus 27 in a functional way, and the support rod 3D is adapted to connect as support function.

In other characteristics of this embodiment, after connecting the command rods 3C to the external apparatus 27, the command rods 3C are adapted to receive and transmit to the functional element 1 force, power and torque provided by the external apparatus 27.

In one characteristic of some embodiment the invention includes de external apparatus 27. In one characteristic of some embodiment, the invention includes the external apparatus 27 and the external apparatus is the adapter 2 to work with a plurality of functional elements 1. In one characteristic of some embodiments, the external apparatus is a robotic arm 27D; in some embodiments the external apparatus is a surgical robot 27H; in some embodiments the external apparatus is a video camera 27C; in some embodiments, the external apparatuses 27 is a laser 27 J; in some embodiments the external apparatuses 27 is a device with servomotors 27K; in some embodiments the external apparatuses 27 is the handle 27E; in some embodiment, the external apparatuses 27 is electric source; in some embodiments the external apparatuses 27 is ultrasonic generator 35; in some embodiments the external apparatuses 27 is power source, but not limited to them.

In other characteristics of this embodiment, the external apparatus 27 is adapted to be replaced quickly during surgery, for example: starting a surgery using a manual external apparatus 27, then switching to a surgical robot arm 27D; or switching to a image processor; or switching to a surgical stapler manipulator 27G, but is not limited to them.

In other characteristics of this embodiment, the removable rod 3F is adapted to be replaced during the surgical procedure, for example: using the tool 8 that is a scissors and changing to an electric scalpel tool 8, remove the functional element 1, remove the command rod 3C and insert in the invention an electric scalpel rod 3 with insulation and an electric scalpel tool 8.

In other characteristics of some embodiments, at least one of said rods 3 works as the tool 8 to perform a surgery. In other characteristics of this embodiment, some rod 3 are the tool 8, for example, an electric scalpel command rod 3C is adapted to be introduced in the invention and used to perform surgery directly on the tissue 25, another example is the command rod 3C is adapted to be used as an ultrasonic scalpel, the blade 33. In other characteristics of this embodiment, the command rod 3C acts as a tool 8.

In another characteristic of this embodiment, the removable rod 3F is adapted to work as an electrical scalpel. In other characteristics of this embodiment, the removable rod 3F is adapted to work as a needle; the removable rod 3F is adapted to work as a heat transmitter; the removable rod 3F is adapted to work as a cold transmitter; the removable rod 3F is adapted to work as a tube; the removable rod 3F is adapted to work as ultrasonic scissors, but is not limited to them.

In other characteristics of this embodiment, the removable rod 3F may be provided with an outer sheathing layer for use with an electric scalpel, with a Teflon layer to drain. In another characteristic of this embodiment, the removable rod 3F is adapted to work as an electric scalpel. In other characteristics of this embodiment, the removable rod 3F is adapted to work with an ultrasonic scissors. In other characteristic of this embodiment, the removable rod 3F is adapted to work as a tube. In other characteristics of this embodiment, the removable rod 3F is adapted to work as LED or fiber optic lighting but is not limited to them.

FIG. 27 is a perspective view of another embodiment of the invention configurated to connect to a robotic arm 27D, according to another embodiment of the invention. In one characteristic of some embodiments, the invention is part of the robotic arm 27D. In this example, the holder 4 is connectable to the robotic arm 27D. In other characteristics of this embodiment, it has four rod 3, three of them being the support rods 3D and one being the command rod 3C. In other characteristics of this embodiment, the protector guide 7 is adapted to run on the rod 3 of the proximal end 3A to the distal end 3B, as indicated by the two-headed arrow.

In other characteristics of this embodiment, two functional elements 1 are available to connect to the distal end 3B. In other characteristics of this embodiment, the functional element 1 is adapted to be used and replaced by another during surgery, the type of functional element 1 that will be used is not limited to them. In another characteristic of this embodiment, the tool 8 that will be used is not limited to them. In another characteristic of this embodiment, the functional element 1 includes the tool 8, the connector 37 and the adapter 2. In one characteristic of some embodiments, there are more than one functional element 1 connectable to the support rod 3D.

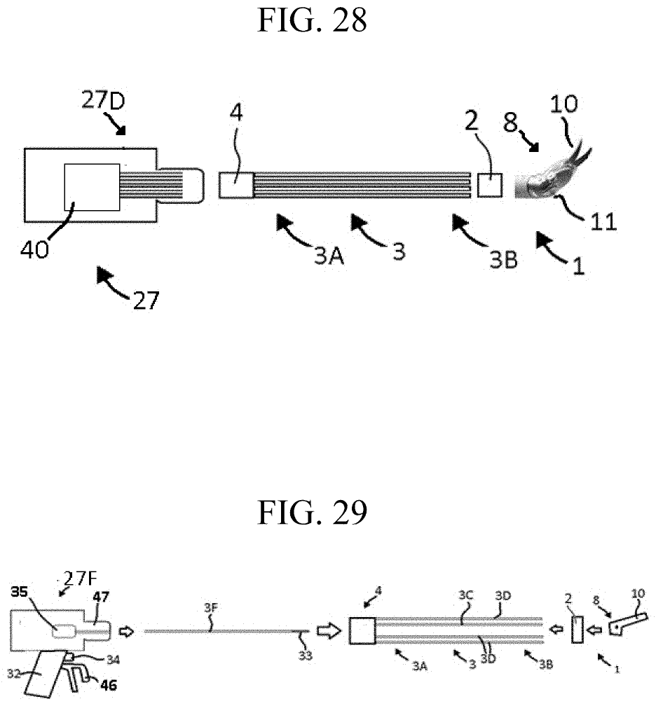

FIG. 28 is a side lateral view of the invention adapted to be robotic operating, according to another embodiment of the invention. In another characteristic, this embodiment is adapted to work with the external apparatus 27, according to another embodiment of the invention.

In another characteristic of this embodiment, the external apparatus 27 is the robotic arm 27D. In other characteristics of this embodiment, the holder 4 is configurated to detachably connect to the robot arm 27D to perform a robotic surgery. In other characteristics of this embodiment, the robot arm 27D has servo motors 40 to actuate in the command rod 3C. In one characteristic of some embodiments, the number of servo motors 40 in the robotic arm 27D is not limited to one.

In another characteristic of this embodiment, the robotic arm 27D is detachably connected to the holder 4 by a quick connection 45.

In another characteristic of this embodiment, the quick connection 45 connects each of the command rod 3C to the servo motors 40 of the robotic arm 27D. In another characteristic of this embodiment, the robotic arm 27D is adapted to transmit, at least one of, force, power and torque to, at least one of, the command rod 3C.

In another characteristic of this embodiment, the robotic arm 27D moves the command rod 3C to manipulate the functional element 1.

In another characteristic of this embodiment, the robotic arm 27D is adapted to use the command rods 3C.

In another characteristic of this embodiment, it is adapted to interchangeably connect to a plurality of the functional elements 1. In another characteristic of this embodiment, the rods 3 controls the functional element 1. In another characteristic of this embodiment, the functional element 1 is modular. In another characteristic of this embodiment, the functional element 1 is modular, including the tool 8 and the adapter 2. In another characteristic of this embodiment, one part the functional element 1 is movable with respect to one another in a first plane and said parts are pivotable relative to said distal end 3B of said shaft in said first plane. In another characteristic of this embodiment, the tool 8 is movable with respect to the adapter 2 in a first plane and are pivotable relative do the distal end 3B in a second plane.

FIG. 29 is the lateral view of the invention, including the ultrasonic scissors, according to another embodiment of the invention.

In this embodiment, the functional element 1 is modular, including the adapter 2, the tool 8 and one jaw 10.

In one characteristics of this embodiment, the functional element 1 is connectable to the distal end 3B. In one characteristics of this embodiment, the holder 4 is connectable to the external apparatus 27. In one characteristics of this embodiment, the removable rod 3F is connectable to the holder 4.

In one characteristics of this embodiment, the holder 4 is adapted to connect to the removable rod 3F, the removable rod 3F is adapted to be inserted in the holder 4. In one characteristics of this embodiment, the tool 8 is adapted to connect to the adapter 2 and the functional element 1 is adapted to connected to the distal end 3B.

In another characteristic of this embodiment, the functional element 1 connects disconnectedly to the distal end 3B.

Another characteristic of this embodiment is that the removable rod 3F is adapted to be disconnected from de holder 4 and replaced by other removable rod 3F during a surgery.

One characteristic of this embodiment is the longitudinal movement of a trigger 46, which drives the command rod 3C in a longitudinal move that drives the jaws 10 to open and close relative to a blade 33 to cut.

In one characteristic of some embodiments of the ultrasonic device 27F, the ultrasonic generator 35 is in the external apparatus 27. In one characteristic of some embodiments, the ultrasonic generator is in the functional element 1.

In other characteristics of this embodiment, a battery 32 is in the external apparatus 27. In one characteristic of some embodiments, the command rod 3C conducts electrical energy to the existing ultrasonic generator 35 in the functional element 1. In another characteristic of this embodiment, the rod 3 is an electric conductor that has an insulating outer layer. Another characteristic is that the ultrasonic generator transmits power, force and torque to the functional elongate rod 3C in order to make the blade 33 the cutting member. In another characteristic of this embodiment, the functional element 1 is adapted to pass through a conventional trocar.

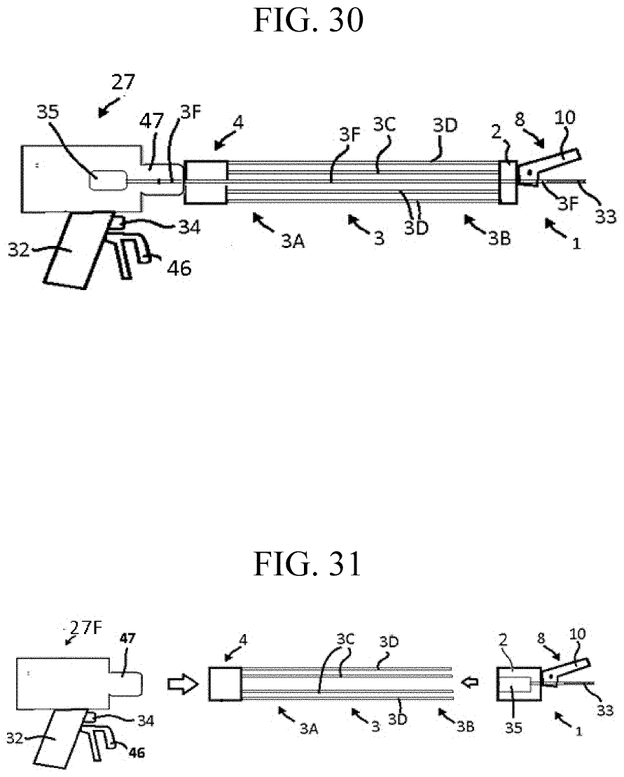

FIG. 30 is a cross-sectional view of another embodiment of the invention, including an ultrasonic scissors. One characteristics of this embodiment, the claw 10 is connected to the adapter 2. One characteristics of this embodiment, the removable rod 3F is connected and ready for use. In one characteristic of this embodiment, a button 34 activates the ultrasonic generator 35 which acts on the removable rod 3F making the blade 33 of the ultrasonic device 27F functional.

Other characteristics of this embodiment, moving forward, the trigger 46 causes the command rod 3C to move the jaws 10 towards the blade 33. One characteristics of this embodiment, the pressing the button 34 drives the ultrasonic generator 35, which uses the energy of the batteries 32 to move the blade 33 to cut.

FIG. 31 is the lateral view of another embodiment of the invention including the ultrasonic scissors 27F. In one characteristics of this embodiment, the holder 4 is connectable to the external apparatus 27. In another characteristic of this embodiment, the external apparatus 27 is an ultrasonic device 27F.

Another characteristic of this embodiment is that the control command rod 3C is adapted to be disconnected from the holder 4 and replaced by other control rod 3 during surgery.

In one characteristics of this embodiment, the functional element 1 is a single piece and is disconnected from the distal end 3B, the larger arrow shows the direction in which the ultrasonic device 27 F connects to the holder 4. In one characteristics of this embodiment, the minor arrow shows the direction in which the functional element 1 connects to the distal end 3B.

One characteristic of this embodiment is the longitudinal movement the lever 4C 46 drives the command rod 3C causing the jaws 10 to open and close relative to the blade 33 to cut.

In other characteristics of this embodiment, in the command rod 3C exists electric wires connecting the battery 32 to the ultrasonic generator 35. In another characteristic of this embodiment, the ultrasonic generator 35 is in the functional element 1. In one characteristic of some embodiments, the battery 32 may be used as the handler. In other characteristic of this embodiment, that actuating the button 34 triggers the ultrasonic generator 35 that drives the blade 33 to cut. In another characteristic of this embodiment, the holder 4 is modular, a first portion 4D connects disconnectedly to a second portion 4E. In another characteristic of this embodiment, the functional element 1 is adapted to pass through a conventional exchange. In another characteristic of this embodiment, the functional element 1 connects disconnectedly to the distal end 3B. In another characteristic of this embodiment, the functional element 1 is modular. In one characteristic of some embodiments, the functional element 1 is a single piece.

In one characteristics of this embodiment, an articulation joint 11 A and an articulation joint 11 B

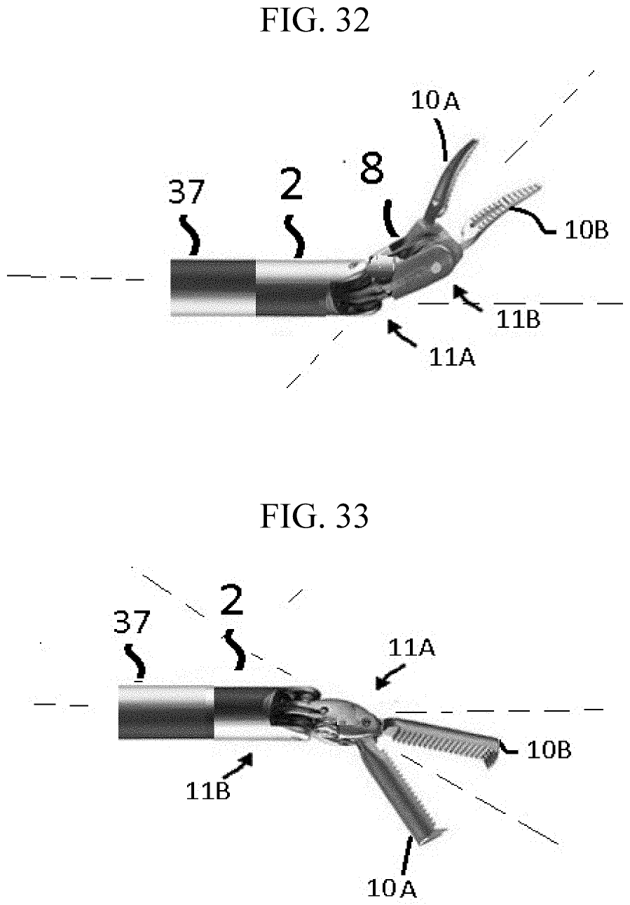

FIG. 32 and FIG. 33 are perspective views of other embodiments of the invention, in which the functional element 1 is articulable rotating, opening and closing its jaws 10A and jaws 10B, according to another embodiment of the invention. In one characteristics of this embodiment, the movement is carried out by means of rotors connected to the rod 3, which connect to the functional element 1 to receive the commands of torque and power of the command rod 3C. In one characteristics of this embodiment, the functional element 1 is controlled from the external apparatus 27. In one characteristics of this embodiment, the tool 8, may be scissors and tweezers, but is not limited to them. In one characteristics of this embodiment, the functional element 1 comprises a pair of cooperating jaws 10A and 10B made to perform a surgical operation. In one characteristics of this embodiment, the functional element 1 further comprises the tool 8 made to perform a surgical operation. In one characteristics of this embodiment, the functional element 1 is made to perform a surgical operation. In one characteristics of this embodiment, the functional element 1 is adapted to interchangeably connect to another functional element.

In one characteristics of this embodiment, the functional element 1 comprises two modular parts. In one characteristics of this embodiment, the is adapted to be robotic operating. In one characteristics of this embodiment, the comprising the articulation joint 11A and the articulation joint 11B. In one characteristics of this embodiment, the functional element 1 is rotatable around two the linear axes, around the axis of the instrument and around the axis of the tool 8. In one characteristics of this embodiment, the functional element 1 includes two movable parts with respect to one another in a first plane and the parts are pivotable relative to said distal end 3B of said shaft in said first plane. In one characteristics of this embodiment, the functional element 1 is modular and detachably connected to said distal end 3B. In one characteristics of some embodiment, the functional element 1 is a surgical stapler.

In one characteristics of this embodiment, the distal end 3B is adapted to interchangeably connect to a plurality of the functional elements 1.

In one characteristics of this embodiment, the holder 4 is adapted to interchangeably connect to a plurality of said external apparatus 27. In some embodiments, the functional element 1 further comprises a needle holder.

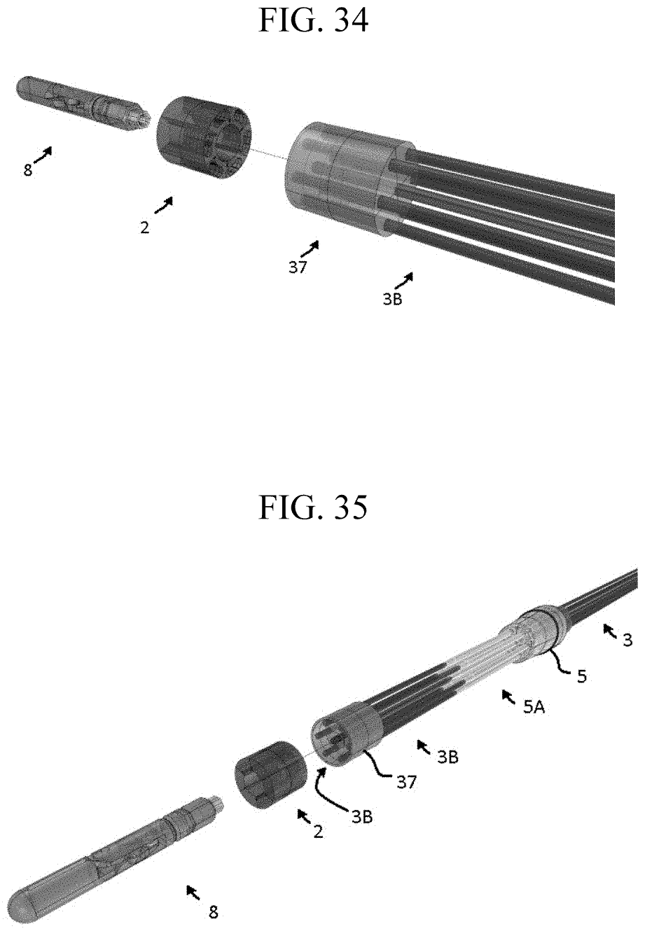

FIG. 34 is a perspective view of the tool 8, the adapter 2 and the connector 37 and the distal end 3B, according to another embodiment of the invention.

This functional element 1 is adapted to be quickly replaced by the adapter 2, allowing the connection of several different types of tool 8. In one characteristics of this embodiment, the tool 8 is adapted to be quickly coupled and disconnected from the adapter 2. In one characteristics of this embodiment, the adaptor 2 is adapted to be quickly replaced with another adapter 2, is adapted to be quickly removed from connector 37 and replaced with another adapter 2.

In some embodiments, the functional element 1 need to use several commands rods 3C for example: the tool 8 that is a stapler or the tool 8 that is an instrument for robotic surgery.

In some embodiments, the functional element 1 needs only one commando rod 3D. In one characteristics of this embodiment, the same command rod 3C opens and closes the jaws 10 with the longitudinal move and rotate the functional element 1 around one axis.

In some embodiments the functional element 1 needs to rotate around two or more axis, in this case the functional element 1 needs more commands rods 3C.

In one characteristics of this embodiment, the connector 37 facilitates the fitting of the adapter 2 in the distal end 3B of the rod 3. In that example, to connect the adapter 2 in the connector 37, the adapter 2 is engaged in the connector 37 and rotated around its axis. To disconnect the adapter 2 from the connector 37, the adapter 2 is rotated around the reverse direction to disconnect the adapter 2 from the connector 37.

FIG. 35 is a perspective view of the invention, including the functional element 1 including the tool 8, the adapter 2, the connector 37, and seven elongate rods 3 which are sectioned to be viewed as the distal end 3B, according to another embodiment of the invention. In one characteristics of this embodiment, the tool 8 is disconnected from the adapter 2. In one characteristics of this embodiment, the connector 37 is connected to the distal end 3B. In one characteristics of this embodiment, the distal rod 3B has seven rod 3.

In one characteristics of this embodiment, the connector 37 is adapted to engage the adapter 2. In that example, to connect the adapter 2 to the connector 37, the adapter 2 is engaged in the connector 37 and rotated. To disconnect the adapter 2 from the connector 37, the adapter 2 is rotated in the reverse direction in order to disconnect the adapter 2 from the connector 37.

In one characteristics of this embodiment, the trocar 5 are used to transfix the tissue. In one characteristics of this embodiment, the cannulas 5A are adapted to cause minimum trauma to the tissue in order to prevent scar. In one characteristics of this embodiment, the cannulas 5A have an outer diameter thin enough to cause minimal trauma to the tissue.

In one characteristics of this embodiment, the rods 3 uses the passageways 28 provided by the trocar 5 to access the surgical site 26.

In one characteristics of this embodiment, the connector 37 is connected to the distal end 3B, inside the surgical site 26, after the elongate rods 3 pass through the trocar 5.

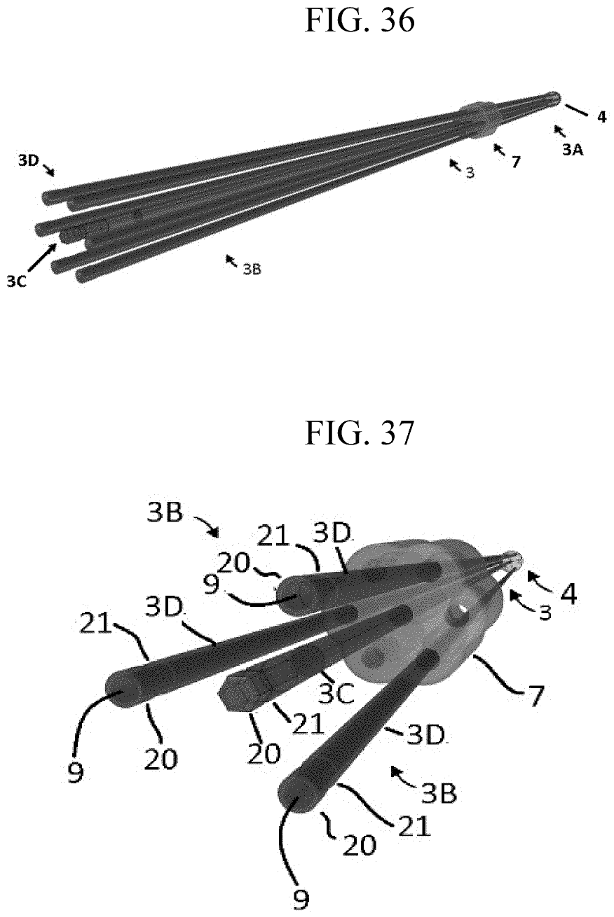

FIG. 36 and FIG. 37 are perspective views of the distal end 3B and the protector guide 7 according to another embodiment of the invention.

In one characteristics of this embodiment, the protector guide 7 is movable longitudinally with respect to the rod 3. In another characteristic of this embodiments, the supports rods 3D are removable. In FIG. 36 there are seven rods 3 and three removable rods 3F. In FIG. 37 three removable rods 3F were removed remaining four rod 3 for use, three rods 3D for support and one command rod 3C to provide force, power and torque to the functional element 1. In one characteristics of this embodiment, the protector guide 7 protect the rods from inadvertently hurting something. In one characteristics of this embodiment, the protector guide 7 guide de replacement of the removable rods 3F. In one characteristics of this embodiment, the protector guide 7 is usually fitted into the slots of a trocar 5, so the rod 3 are pushed into the trocar 5 guided by the protector guide 7. In one characteristics of this embodiment, the protector guide 7 serves as a guide to prevent them from stepping out of the rods 3. In one characteristics of this embodiment, the rod 3 have a slightly smaller diameter than the passageways 28 of the cannulas 5A, to be able to move longitudinally inside the is cannulas 5A. In some embodiments there are two protector guide 7. In one characteristics of some embodiment, the second protector guide 7 facilitates the removal of the rod 3 and the replacement of the rod 3.

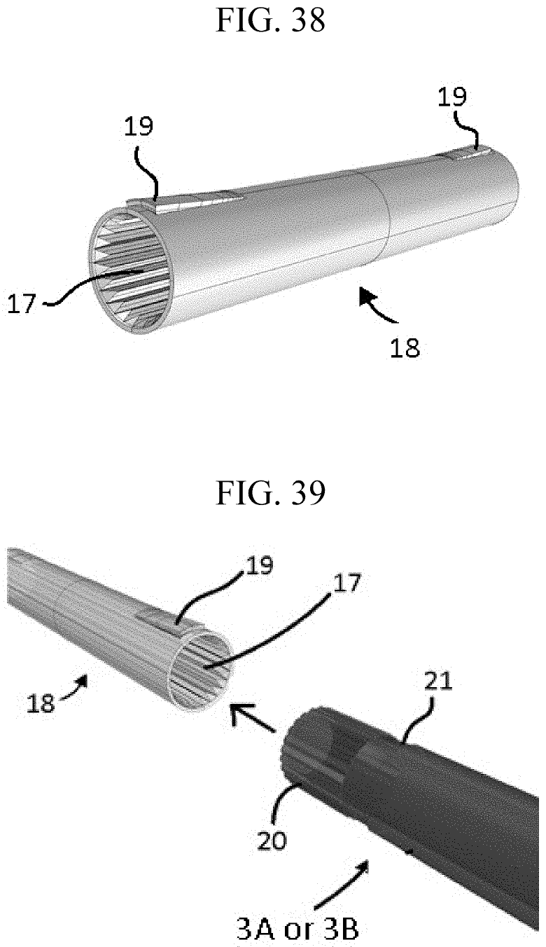

FIG. 38 and FIG. 39 are perspectives views of a link 18 connection according to another embodiment of the invention. In one characteristics of this embodiment, the link 18 allows to quickly connect two rods 3. In one characteristics of this embodiment, the distal end 3B enters and snaps into the socket 17. In one characteristics of this embodiment, a lock 19 of the link 18 prevents the elongate rod 3 from leaving. In one characteristics of this embodiment, the link 18 stays inside a housing 12. To connect the rod 3, the link 18 it is pulled out of a housing 12 until the lock 19 rises. In one characteristics of this embodiment, the rod 3 is docked and is pushed back to the housing 12 the lock 19 and the rod 3 is connected. In one characteristic of this embodiment, the lock 19 with the connected rod 3 moves inside a socket 17 to transmit torque and power force to the other rod 3. In one characteristics of this embodiment, the link 18 is where the rod 3 connects. In one characteristics of this embodiment, the link 18 allows the distal end 3B to be connectable to the holder 4 in a disconnecting manner. In one characteristics of this embodiment, the link 18 also connects the distal end 3B with the functional element 1 in a disconnect able manner. In one characteristics of this embodiment, the lock 19 engages a slit 21 to disconnectedly connect the rod 3 to the link 18. In one characteristics of this embodiment, the arrow shows the direction in which the rod 3 engages a gear 20.

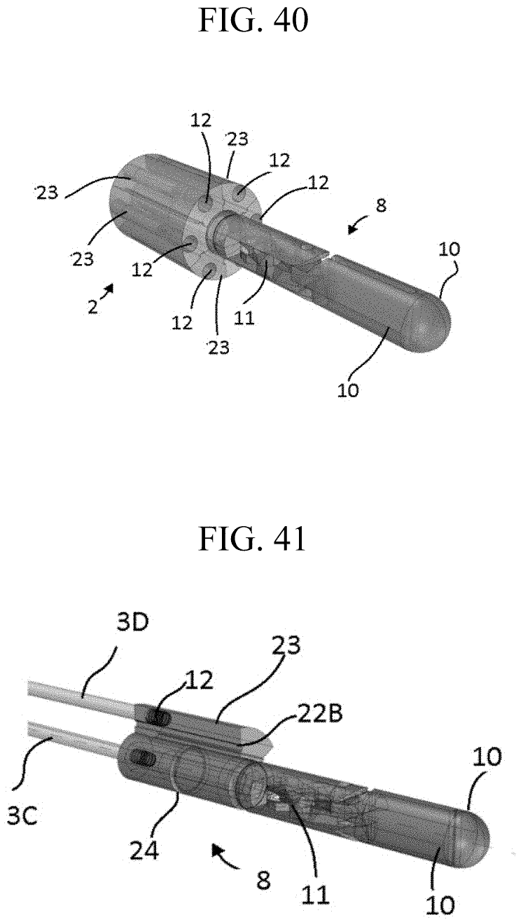

FIG. 40 is a perspective view of the functional element 1, according to another embodiment of the invention.

Various types of tool 8 are adapted to be inserted in the housing 12. In one characteristics of this embodiment, the support rod 3D is adapted to connect in the housing 12 to a cart 23. In one characteristics of this embodiment, the control rod 3C is connected to the tool 8. In the tool 8 there is a groove 24 which allows the tool 8 to rotate relative to its axis. In one characteristics of this embodiment, the articulation joint 11 is actionable by the command rod 3C to open and close the pair of jaws 10. In one characteristics of this embodiment, the housing 12 is also used to connect other types of tool 8 to adapter 2. In one characteristics of this embodiment, the adapter 2 is adapted to be connect several types of tip tools 8. In one characteristics of this embodiment, the tool 8 is connected to the adapter 2 so that it is adapted to rotate around the adapter 2 axis. In one characteristics of this embodiment, the adapter 2 is adapted to be docked to the distal end 3B with the aid of the connector 37.

FIG. 41 This is a perspective view of a portion of the functional element 1 directly connected to the distal end 3B, according to another embodiment of the invention.

Only one card 23 is seen, the other carts 23 have been removed for easy viewing. In one characteristics of this embodiment, the card 23 is connected to a support's rod 3D. In one characteristics of this embodiment, the command rod 3C is adapted to move longitudinally in the manner to drive the articulation joint 11. In one characteristics of this embodiment, the grove 24 allows the tool 8 to rotate without moving in the longitudinal direction. This allows the articulation joint 11 to be fired in order to open and close the jaws 10. In this embodiment the female rail 22B is seen.

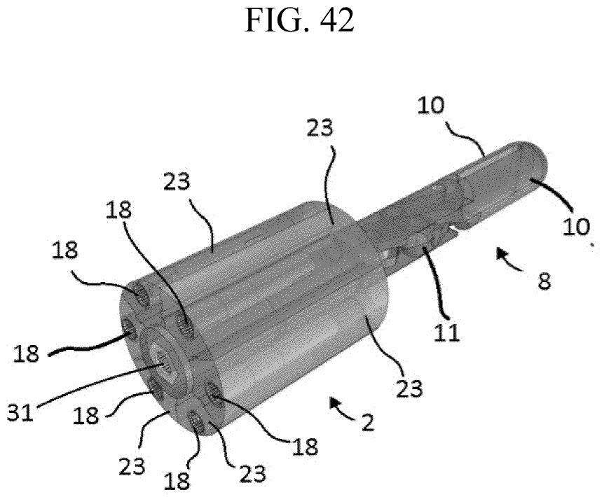

FIG. 42 is another embodiment of the invention that uses the link 18 to connect the rod 3 to the adapter 2. In one characteristics of this embodiment, the card 23 connects the command rod 3C that opens and closes the jaws 10 and rotates the tool 8. In other characteristic this embodiment, the cart 23 is adapted to work as a support function or as a control function. In one characteristics of this embodiment, the link 18 is adapted to transmit power, force a torque to the tool 8. In one characteristics of this embodiment, the card 23 is adapted to be made of an electrical insulator material to connect to a rod 3 that has a layer of insulation. In other characteristic this embodiment, the card 23 is adapted to be in electrical connection with the functional element 1. In other characteristics of this embodiment, at least one of said rods 3 is adapted to be in electrical connection with the functional element 1.

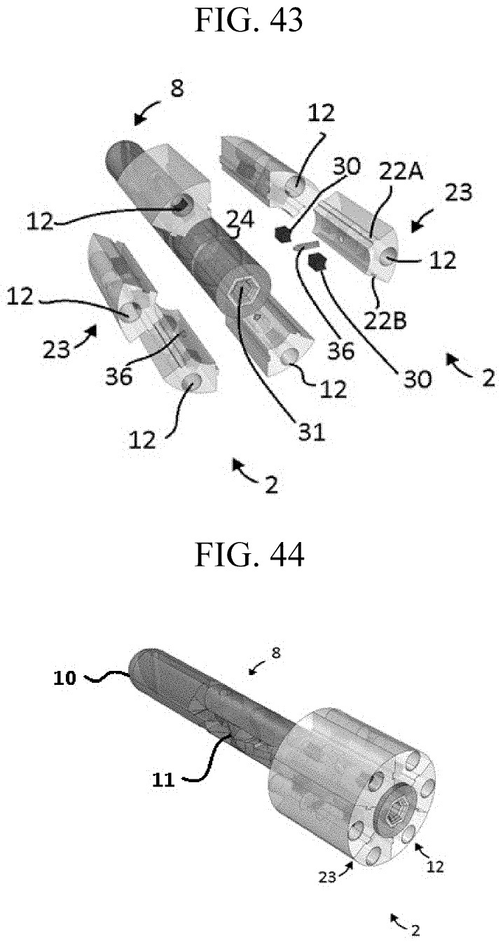

FIG. 43 is an exploded perspective view of the functional element 1, according to another embodiment of the invention.

In one characteristics of this embodiment, a pin 36 engages the groove 24 and prevents the tool 8 from moving longitudinally while allowing it to rotate around its axis. In one characteristics of this embodiment, a female-connecter 31 is where the command rod 3C connect to open and close the jaws 10. In one characteristics of this embodiment, a beater 30 keeps the link 18 in the housings 12.

In one characteristics of this embodiment, the male rail 22A and the female rail 22B allow longitudinal displacement of the carts 23 and at the same time keeps one cart 23 connected to the other cart 23 forming a cohesive carton.

FIG. 44 is another view of the functional element 1 connected to an adapter 2, according to another embodiment of the invention.

It is adapted to be connected directly to the distal end 3B.

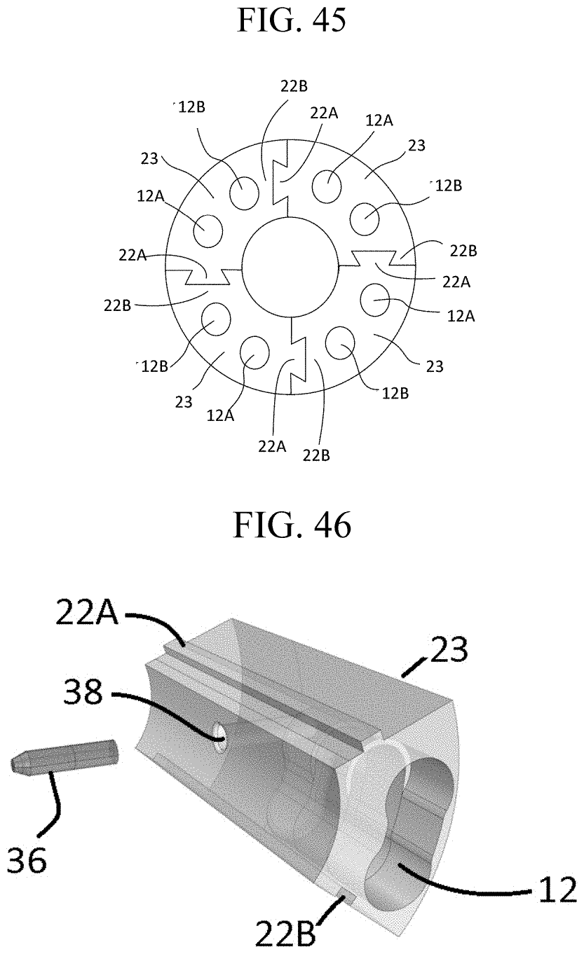

FIG. 45 It is a trans sectional view of the adapter 2 with four cart 23, according to another embodiment of the invention.

In one characteristics of this embodiment, the number of carts 23 is not limited to these. In other characteristic this embodiment, the cart 23 has a housing 12A and another housing 12B. In one characteristics of this embodiment, the carts 23 have male rails 22A and female rails 22B, one slides longitudinally over the other.

FIG. 46 is a perspective view of the cart 23 for quick connection 45, according to another embodiment of the invention.

In another characteristic this embodiment, the cart 23 is adapted to connect to the rod 3. In one characteristics of this embodiment, the pin 36 is adapted to enter in the basement 38. In one characteristics of this embodiment, the housing 12 is made to receive the distal end 3B. In one characteristics of this embodiment, each the distal end 3B are adapted to engages in the housings 12 of the adapter 2. In one characteristics of this embodiment, the adapter 2 is rotated by securing the distal end 3B in the housing 12.

In the housing 12 the distal end 3B of the rod 3 pushes the pin 36 that enters the groove 24 that holds the tool 8, the way to connect the tool 8 is not limited to that. Generally, the pin 36 remains in the basement 38, moves in but is adapted to not come out.

It is the example of a carton adapted for quick connection 45 by rotation the rod 3 is engaged in the housing 12 and moves from one position to the other position inside the housing 12, to disconnect is needed to rotate de adapter 2 in the opposite direction.

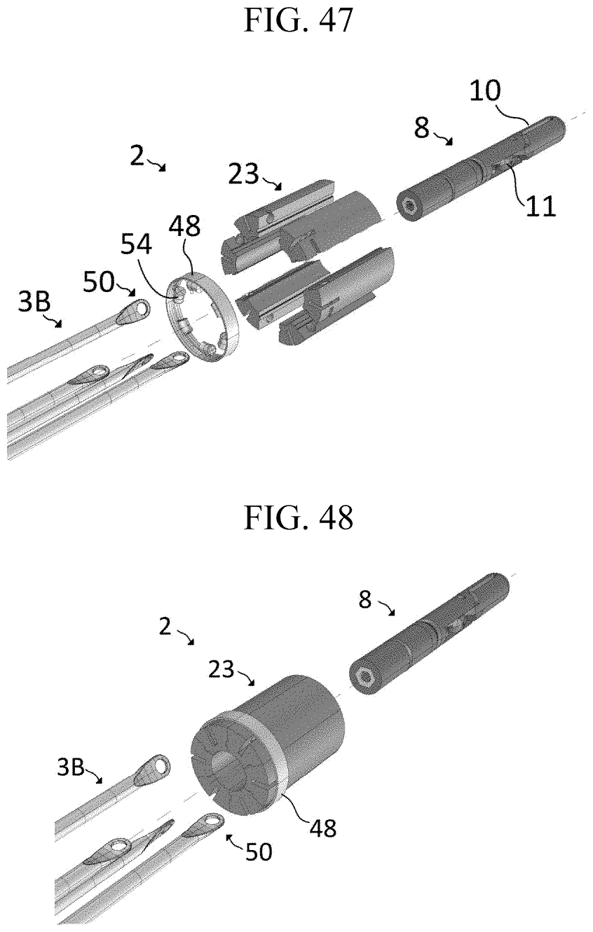

FIG. 47 and FIG. 48 are perspective views of the quick connection 45 according to another embodiment of the invention.

In this embodiment, the rod 3 has a hollow 50 in their distal end 3B with a rotating ring 48. A tongue 54 engages inside the hollow 50 by securing the rod 3. In one characteristics of this embodiment, the rod 3 is released by rotating the ring 48 in the opposite direction.

In some embodiments, there is a specially adapted device to rotate the rotating ring 48.

FIG. 49 is a perspective view of the quick connection 45, according to another embodiment of the invention.

In this embodiment, the rods 3 are threaded at their distal end 3B.

In one characteristics of this embodiment, the rods 3 connect to the adapter 2 by rotation and the cart 23 have a female thread 58 in the housing 12 for the rod 3 to screw 60.

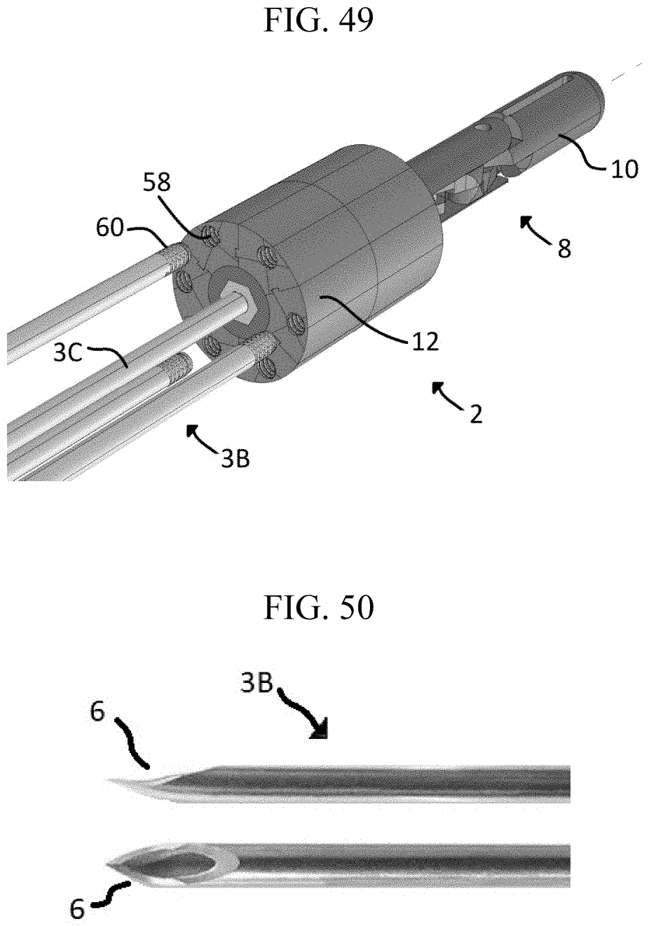

FIG. 50 is a lateral view of the distal end 3B including the piercing tip 6, according to another embodiment of the invention.

In one characteristic of this embodiments, each the rods 3 distal end 3B have a piercing tip 6 to perforate de tissue 25. In other characteristic of this embodiment, the piercing tips 6 are adapted do connect to the functional element 1. In other characteristics of this embodiment, the piercing tips 6 are adapted to leave no scar on the skin. In other characteristics of this embodiment, the piercing tips 6 are adapted to cause minimal trauma to the tissue 25 in order not to leave any scarring in the tissue 25. In other characteristics of this embodiment, the functional element 1 is adapted to connect to the piercing tip 6 rod 3. In other characteristics of this embodiment, each of the rod 3 further includes the piercing tip 6 in the distal end 3B to puncture the tissue 25. In other characteristics of this embodiment, the piercing tip 6 have a sharp tip to easily penetrate the tissue 25 and cause no scars. In other characteristics of this embodiment, the smaller the external diameter of the elongate rod 3, lower is the risk of scarring by perforation.

FIG. 51 is a lateral view of the distal end 3B of the rod 3, including four trocars 5 and four cannulas 5A, according to another embodiment of the invention.

In other characteristics of this embodiment, the surgical device for insertion into a surgical site 26 through a plurality of perforations in a tissue 25, including: a plurality of rod 3, the rod 3 including a proximal end 3A and a distal end 3B, and a holder 4 connected to the proximal end 3A, further comprising at least one trocar, further comprising piercing tip 6. In other characteristics of this embodiment, the piercing tip 6 has a sharp tip to easily penetrate the tissue 25 and cause no scars. In other characteristics of this embodiment, the smaller the external diameter of the cannulas 5A, lower is the risk of scarring by perforation. In other characteristics of this embodiment, the cannulas 5A have an external diameter thin enough to cause minimal trauma to the tissue 25.

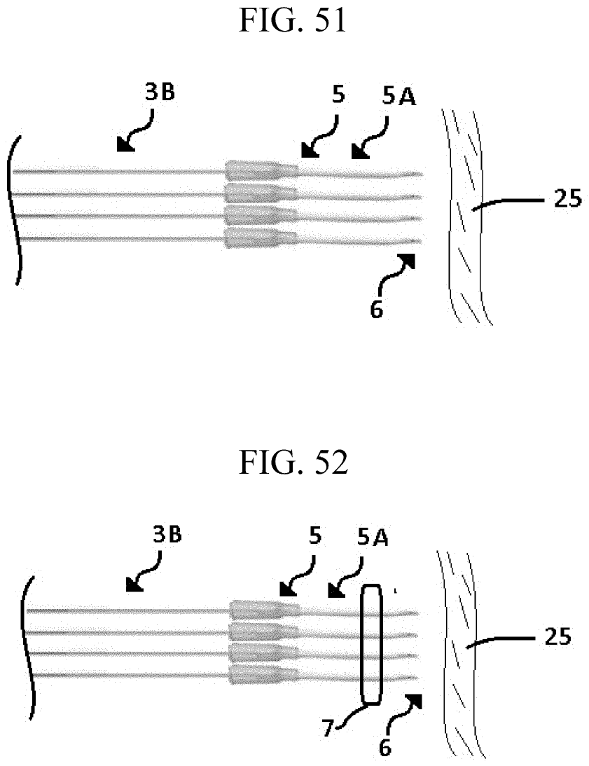

FIG. 52 is a lateral view of the distal end 3B including a plurality of trocars 5 and a protector guide 7, according to another embodiment of the invention. In another characteristic of this embodiment, there's a protector guide 7 on the cannulas 5A. In another characteristic of this embodiment, the protector guide 7 slides along the cannulas 5A. In another characteristic of this embodiment, the protector guide 7 protects the cannulas 5A and directs the insertion of the piercing tip 6 into the tissue 25. In another characteristic of this embodiment, the protector guide 7 drives the insertion of the rod 3 into the tissue 25. In another characteristic of this embodiment, the protector guide 7 holds the cannulas 5A together. In another characteristic of this embodiment, the protector guide 7 is adapted to not delimit out of the rods 3. In another characteristic of this embodiment, the protector guide 7 protects the piercing tip 6 so as not to hurt something. In another characteristic of this embodiment, the protector guide 7 drive is adapted to not slide out of the rod 3. One characteristic of this embodiment is that each of the rod 3 have an outer diameter adapted to cause minimal trauma to the tissue 25, in order to prevent scarring. In one characteristics of this embodiment, the rod 3 outer diameter are thin enough to access a surgical site 26 and cause only minimal trauma to the tissue 25.

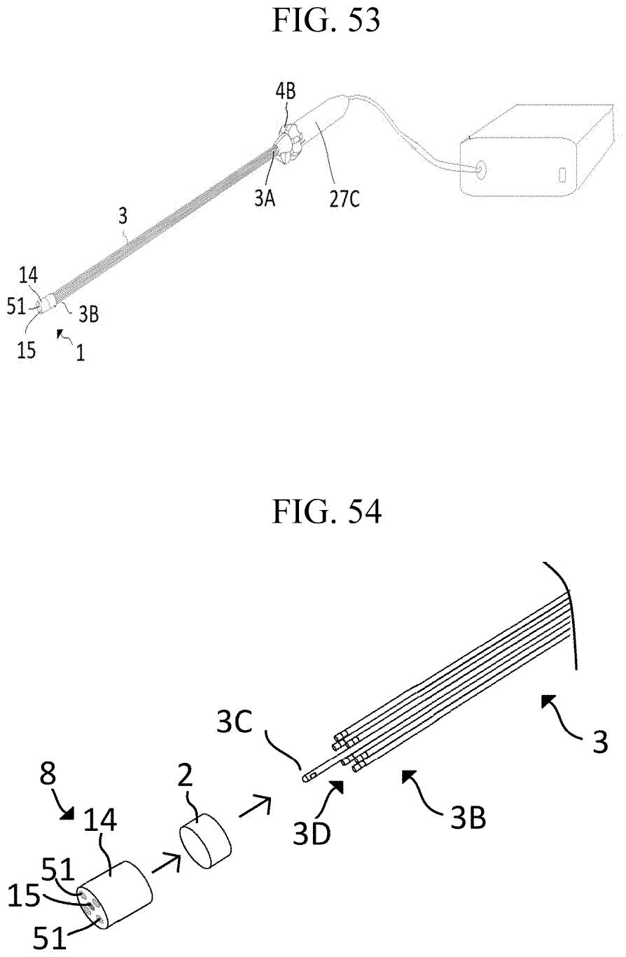

FIG. 53 is a perspective view of the video camera 27C, according to another embodiment of the invention.

In other characteristics of this embodiment, there is a surgical device for insertion into a surgical site 26 through a plurality of perforations in a tissue 25, comprising: a plurality of rods 3, the rods 3 including proximal ends 3A and distal ends 3B, a holder 4 connected to the proximal ends 3A and the functional element 1 connectable to the distal end 3B. In another characteristic of this embodiment, there is further comprising at least one video camera 27C.

In another characteristic, some embodiment of the invention further comprises at least one video camera 27C.

In other characteristics of this embodiment, the functional element 1 further comprising at least one camera head 14.

FIG. 54 is a perspective view of the functional element 1, including a camera heard 14, according to another embodiment of the invention.

In one characteristics of this embodiment, the camera head 14 is disconnected from the adapter 2 and from the distal end 3B. An optical path 13 extends through the lumen 9A of the rod 3, connected to a light source 27B to illuminate the surgical site 26. In other characteristics of this embodiment, the camera head 14 electronics includes an imager 29 (it is not shown in the drawing) to produce an electronic image, and a power source (it is not shown in the drawing).

This embodiment is adapted to operatively connect to at least one external apparatus 27 including: an image processor 27A, a video camera 27C and a light source 27B.

In other characteristics of this embodiment, the electrical wires are adapted to connect the camera head 14 to an external apparatus 27, which is an image processor 27 A. In another characteristic of this embodiment, the image is adapted to be displayed on a video monitor (it is not shown in the drawing). In other characteristics of some embodiment, the image processor 27A is a control circuitry for processing the electronic image as produced by the imager 29.

In another characteristic of this embodiment, it includes the imager 29 for image pickup. Also, another characteristic of some embodiment, is that electrical wires in the lumen 9A communicate the imager 29 with the image processor 27A. In another characteristic of this embodiment, the imager 29 is adapted to connect to the electrical wires.

In another characteristic of this embodiment, the imager 29 is a solid-state imager 29. In another characteristic of some embodiment, the imager 29 is a CCD imager 29. In another characteristic of some embodiment, the imager 29 is a CMOS imager 29. In other characteristics of some embodiment, the imager 29 and camera head 14 are configurated on a single chip. In other characteristics of this embodiment, the imager 29 produces an electronic image upon being actuated by the image processor 27A control circuitry.

A characteristic of some embodiment is that the lumen 9A of the rod 3 includes a first optical path 13A. In another characteristic of some embodiment, the first optical path 13A extends through the rod 3 and through the holder 4.

In other characteristics of some embodiment, the second optical path 13B extends through the rod 3 and through the holder 4. In another characteristic of some embodiment, the second optical path 13B extends from the light source 27B to the camera head 14,

In one characteristic of some embodiments, there is a camera heard 14 including the two imagers 29. In one characteristic of some embodiments, there is a camera heard 14 including three imagers 29, but the number of imagers 29 in the camera heard 14 are not limited to them. In another characteristic of some embodiment, the second optical path 13B has optical fibers that pass through a lumen 9B. In other characteristics of this embodiment, a second optical path 13 B is implemented with a liquid light guide. In other characteristics of some embodiment, the lumen 9 B of the rod 3 is an optical path extending from the light source 27B to the camera head 14.

In other characteristics of some embodiment, there is an external apparatus 27 which is a light source 27B. In other characteristics of some embodiment, there is a connection from the light source 27B to the existing optical fibers inside the lumen 9B to illuminate the surgical site 26. In one characteristic of some embodiments, there are two optical paths 13 to illuminate the surgical site 26. In one characteristic of some embodiments, there are three optical paths 13 to illuminate the surgical site 26, but the number of optical paths 13 to illuminate the surgical site 26 are not limited to them. In one characteristics of some embodiment, the number of rods 3 used to illuminate the surgical site 26 is not limited to them.

In another characteristic of this embodiment, the rod 3 includes optical lenses 15 (it is not shown in the drawing) at the functional element 1. In another characteristic of some embodiment, the lumen 9A has optical lenses 15 for transmitting images of the surgical site 26 to an image processor 27A. In another characteristic of some embodiment, the lumen 9A has optical lenses 15 for transmitting images of the surgical site 26 to a camera head 14. In another characteristic of some embodiment, it captures the images of the optical lenses of the lumen 9 with an external apparatus 27, which is a video camera 27C, and displays the images of the surgical site 26 on a monitor. In another characteristic of some embodiment, it captures the images of the optical lenses 15 of the lumen 9 with an external camera head 14 which is an external apparatus 27A.

It will be apparent to one skilled in the art that the invention may be provided including some or all the mentioned features and components without departing from the spirit and scope of the invention. For purposes of comparing some embodiments, certain aspects and advantages of these embodiments are described. Not necessarily all such aspects or advantages are achieved by any particular embodiment. Thus, for example, some embodiments can be carried out in a manner that achieves or optimizes one advantage or group of advantages as described, herein without necessarily achieving other aspects or advantages as can also be described or suggested herein. It will also be apparent to the skilled artisan that the embodiments described above are specific examples of a broader invention which may have greater scope than any of the singular descriptions taught. There may be many alterations made in the descriptions without departing from the spirit scope of the invention. Although, certain preferred embodiments and examples are disclosed, inventive subject matter extends beyond the specifically disclosed embodiments to other alternative embodiments and/or uses, and to modifications and equivalents thereof. Thus, the scope of the claims appended hereto is not limited by any of the particular embodiments described. For example, in any method or process disclosed herein, the acts or operations of the method or process can be performed in any suitable sequence and they are not necessarily limited to any particular disclosed sequence. Some operations can be described as multiple discrete operations in turn, in a manner that can be helpful in understanding certain embodiments, however, the order of description should not be construed to imply that these operations are order dependent.

I claim:

1. A video-surgical instrument, comprising: (a) a plurality of elongated rods, each including a proximal end and a distal end; (b) a holder; (c) a functional element; and (d) a fairing; wherein each said proximal end is connectable to said holder; wherein each said distal end is connectable to said functional element; wherein said fairing removably covers said plurality of elongated rods.
1.1. The surgical instrument of claim 1, wherein said holder is in connection with said proximal end to transmit at least one of force, power and torque, to said rods.
1.2. The surgical instrument of claim 1, wherein said fairing covers said rods in a sealing manner.
1.3. The surgical instrument of claim 1, wherein said holder is adapted to interchangeably connect to a plurality of external apparatus, to transmit at least one of force, power and torque, to at least one of said rods.
1.4. The surgical instrument of claim 1, wherein at least one of said rods includes at least one lumen.
1.5. The surgical instrument of claim 1, wherein at least one said distal end further comprises a piercing tip.
1.6. The surgical instrument of claim 1, further comprising at least one protector guide.
1.7. The surgical instrument of claim 1, wherein said holder further comprises at least one interconnectable modular part.
1.8. The surgical instrument of claim 1, wherein said holder is connectable to at least one external apparatus for a transmission of at least one of force, power and torque, to at least one said elongated rods, wherein at least one of said elongated rods is a transmitter of at least one of, force, power and torque, for said functional element.
1.9. The surgical instrument of claim 1, wherein said holder is operatively connected to at least one of said rods and at least one of said rods is operatively connected to at last one said functional element.
1.10. The surgical instrument of claim 1, wherein at least one of said rods is interchangeable with other rods.
1.11. The surgical instrument of claim 1, wherein said functional element is made to perform a surgical operation.
1.12. The surgical instrument of claim 1, further comprising electrical connections.
1.13. The surgical instrument of claim 1, wherein said functional element is adapted to interchangeably connect to another functional element inside the surgical site.
1.14. The surgical instrument of claim 1, further comprising at least one modular part.
1.15. The surgical instrument of claim 1, wherein is adapted to be robotic operating.
1.16. The surgical instrument of claim 1, further comprising at least one articulation joint.
1.17. The surgical instrument of claim 1, wherein said functional element is rotatable around one or more axes.
1.18. The surgical instrument of claim 1, wherein said functional element include a camera heard.
1.19. The surgical instrument of claim 1, wherein said instrument is an ultrasonic surgical instrument.
1.20. The surgical instrument of claim 1, wherein said instrument is a surgical stapler.
1.21. The surgical instrument of claim 1, wherein said instrument is adapted to cause minimal trauma to the tissue in order to prevent scar.

2. A method of performing a surgery, comprising the steps of: (a) inserting a fairing to cover a plurality of elongated rods of a video-surgery instrument; (b) inserting said instrument in a surgical site through a conventional trocar; (c) using said instrument to perform a surgery.
2.1. The method in claim 2, further comprising interchanging a functional element of said video-surgical instrument to another said functional element inside said surgical site.

3. A method of performing a surgery, comprising the steps of: (a) removing a fairing to uncover a plurality of elongated rods of a video-surgery instrument; (b) inserting each said plurality of elongated rods in a surgical site through a trocar comprising a plurality of cannulas; and (c) using said instrument to perform a surgery.
3.1. The method in claim 3, further comprising interchanging at least one said rods with another said rod.
3.2. The method in claim 3, further comprising connecting a functional element to said plurality of elongated rods distal end;
3.3. The method in claim 3, further comprising connecting an external apparatus to said plurality of rods proximal end.
3.4. The method in claim 3, further comprising interchanging said functional element to another said functional element inside said surgical site.
